SEQUENCE LISTING (8 sequences)

SEQ ID NO: 1 - protein, 492 aa, Homo sapiens
  MISC_FEATURE: FRS3 from human
MGSCCSCLNRDSVPDNHPTKFKVTNVDDEGVELGSGVMELTQSELVLHLHRREAVRWPYLCLRRYGYDSNLFSFESGRRCQTGQGIFAFKCSRAEEIFNLLQDLMQCNSINVMEEPVIITRNSHPAELDLPRAPQPPNALGYTVSSFSNGCPGEGPRFSAPRRLSTSSLRHPSLGEESTHALIAPDEQSHTYVNTPASEDDHRRGRHCLQPLPEGQAPFLPQARGPDQRDPQVFLQPGQVKFVLGPTPARRHMVKCQGLCPSLHDPPHHNNNNEAPSECPAQPKCTYENVTGGLWRGAGWRLSPEEPGWNGLAHRRAALLHYENLPPLPPVWESQAQQLGGEAGDDGDSRDGLTPSSNGFPDGEEDETPLQKPTSTRAAIRSHGSFPVPLTRRRGSPRVFNFDFRRPGPEPPRQLNYIQVELKGWGGDRPKGPQNPSSPQAPMPTTHPARSSDSYAVIDLKKTVAMSNLQRALPRDDGTARKTRHNSTDLPL

SEQ ID NO: 2 - protein, 491 aa, Mus musculus
  MISC_FEATURE: FRS3 from mouse
MGSCWSCLDRDSVPHNHPTKFKVTNVDDEGVELGSGVMELTQSELVLHLHQREAVRWPYLCLRRYGYDSNLFSFESGRRCQTGQGIFAFKCSRAEDIFNLLQDLMQCNSINVTEEPVIITRSSHPPELDLPRGPPQPAGYTVSGFSNGFPGCPGEGPRFSAPRRPSTSSLRHPSPGEESTHTLIASEEQSHTYVNTPTGDEDGRSRHCLQPLPEGRVPLPAQTQGSDQRDPQVLLQPGQVKFVLGPTPARRQVMKCQSLCPGMQDPPHHNNNEGPSECPAQPKCTYENVSGGLQQGAGWRLSPEERGWSGLAHRRAALLHYENLPPLPPVWESQGQQPGGEAGDDGDSRDGLTPSSNGFPDGEEDETPLQKPTSTRASARSHSGFPVPLTRRRGSPRVFNFDFRRQGPEPPRQLNYIQVELKGWGTARPKGPQNPSVSGAPGPTPHPVRSSDSYAVIDLKKTAAMSDLQRALPRDDGAVRKTRHNSTDLPL

SEQ ID NO: 3 - protein, 508 aa, Homo sapiens
  MISC_FEATURE: FRS2 from human
MGSCCSCPDKDTVPDNHRNKFKVINVDDDGNELGSGIMELTDTELILYTRKRDSVKWHYLCLRRYGYDSNLFSFESGRRCQTGQGIFAFKCARAEELFNMLQEIMQNNSINVVEEPVVERNNHQTELEVPRTPRTPTTPGFAAQNLPNGYPRYPSFGDASSHPSSRHPSVGSARLPSVGEESTHPLLVAEEQVHTYVNTTGVQEERKNRTSVHVPLEARVSNAESSTPKEEPSSIEDRDPQILLEPEGVKFVLGPTPVQKQLMEKEKLEQLGRDQVSGSGANNTEWDTGYDSDERRDAPSVNKLVYENINGLSIPSASGVRRGRLTSTSTSDTQNINNSAQRRTALLNYENLPSLPPVWEARKLSRDEDDNLGPKTPSLNGYHNNLDPMHNYVNTENVTVPASAHKIEYSRRRDCTPTVFNFDIRRPSLEHRQLNYIQVDLEGGSDSDNPQTPKTPTTPLPQTPTRRTELYAVIDIERTAAMSNLQKALPRDDGTSRKTRHNSTDLPM

SEQ ID NO: 4 - protein, 508 aa, Mus musculus
  MISC_FEATURE: FRS2 from mouse
MGSCCSCPDKDTVPDNHRNKFKVINVDDDGNELGSGVMELTDTELILYTRKRDSVKWHYLCLRRYGYDSNLFSFESGRRCQTGQGIFAFKCARAEELFNMLQEIMQNNSINVVEEPVVERSSHQTELEVPRTPRTPTTPGLGAQNLPNGYPRYPSFGDASSHPSSRHPSVGSARLPSVGEESTHPLLVAEEQVHTYVNTTGVQEERKNRASVHVPPEARVSNAESNTPKEEPSNPEDRDPQVLLKPEGVRFVLGPTPVQKQLMEKEKLEQLGKDPVSGSGAGNTEWDTGYDSDERRDVPPVNKLVYENINGLSIPSASGVRRGRLTSTSTSDTQNINNSAQRRPALLNYENLPSLPPVWEARKLSRDEDDNLGPKTPSLNGYHNNLDPMHNYVNTENVTVPASAHKIDYSKRRDCTPTVFNFDIRRPSLEHRQLNYIQVDLEGGSDSDNPQTPKTPTTPLPQTPTRRTELYAVIDIERTAAMSNLQKALPRDDGTSRKTRHNSTDLPM

SEQ ID NO: 5 - DNA, 18 bp, artificial
  synthetic DNA primer
agccacccaatgctctag

SEQ ID NO: 6 - DNA, 24 bp, artificial
  synthetic DNA primer
gtgggggcaggttctcatagtgcg

SEQ ID NO: 7 - DNA, 24 bp, artificial
  synthetic DNA primer
agccacccaatgctctaggctaca

SEQ ID NO: 8 - DNA, 24 bp, artificial
  synthetic DNA primer
gtgggggcaggttctcatagtgca

(19) United States
(12) Patent Application Publication
(10) Pub. No.: US 2007/0274971 A1
(43) Pub. Date: Nov. 29, 2007

(54) Method of Proliferating Precursor Cells

(76) Inventors: Susan Oriole Meakin, London (CA); Kathryn Elizabeth Volkening, West Lorre (CA)

Correspondence Address: CLARK & ELBING LLP, 101 FEDERAL STREET, BOSTON, MA, 02110, US

(21) Appl. No.: 10/591,741

(22) PCT Filed: Mar. 4, 2005
(86) PCT No.: PCT/CA05/00345

§ 371 Date: Jul. 23, 2007

Related U.S. Application Data
(60) Provisional application No. 60/549,870, filed on Mar. 4, 2004.

(51) Int. Cl. A61K 35/12; A61P 25/28; A61P 35/02; C12N 15/86; C12N 5/02; C12N 5/10

(52) U.S. Cl. 424/93.21; 424/93.7; 435/375; 435/455

(57) ABSTRACT

There is provided a method of proliferating precursor cells by modulating the expression of the protein FRS3 within the precursor cells. The method is useful for the production of large populations of precursor cells, which are useful for in vitro research techniques, including drug screening, and for in vivo therapeutic uses.

### CROSS-REFERENCE TO RELATED APPLICATION

This application claims the benefit of U.S. provisional patent application No. 60/549,870, filed on Mar. 4, 2004, which is hereby fully incorporated herein by reference.

### FIELD OF THE INVENTION

The present invention relates generally to precursor cells, and particularly to methods of proliferating precursor cells.

### BACKGROUND OF THE INVENTION

A wide range of human disorders and diseases arise from the premature death or malfunction of specific cell types and the body's failure to replace or restore such cells. Such disorders and diseases include cancers such as leukemia, neurological diseases such as Parkinson's Disease, Alzheimer's and ALS (Lou Gehrig's Disease), CNS damage, cardiac damage, liver damage and diabetes. Typically, current treatments for such disorders and diseases tend to delay the onset of disease and relieve the symptoms of ill health caused by the disease. Although some disorders and diseases can be addressed with transplant surgery, not enough donors exist to treat all patients and, even when rare donors can be found, this is limited to specific tissues and organs and is very expensive.

For example, CNS disorders have been treated with pharmaceutical compounds with varying degrees of efficacy. However, there are a multitude of CNS disorders that continue to be untreatable using conventional methods and compounds. Cell therapy approaches, in comparison to the traditional treatments, look to replace defective cells instead of treating the disease effects. In the past, cell therapy approaches have used primary cell replacement strategies, which involve transplantation of exogenous tissue and activation of proliferation of endogenous cells. However, scarcity of human material, the lack of proliferative capacity of primary cell cultures, and problems associated with the aberrant characteristics that immortalized cell lines often display underscore the need for a dependable source of precursor cells whose proliferation does not affect cell phenotype.

A precursor cell is a cell derived from a fetus or an adult which is partially undifferentiated, and which is capable of either dividing to form additional precursor cells, or dividing to form differentiated cells. Precursor cells are unique in their ability to proliferate for extended periods of time and to differentiate under certain conditions into specialized cell types. Due to their distinctive characteristics, precursor cells offer unique opportunities to develop new therapeutic approaches for many diseases that are currently incurable.

Thus, precursor cell technology, which seeks to treat many degenerative diseases including CNS disorders, is currently being investigated and developed. Approaches are currently being developed which use transplantation of precursor cells as a possible alternative to traditional treatments, since precursor cells can be collected, grown and stored to provide a plentiful supply of healthy replacement cells for transplantation into most sites in the body, using much less invasive surgery than conventional transplants. Approaches that are being developed include using transplanted tissue to directly replace lost tissue, or implanting genetically engineered cells that secrete factors to promote cell survival and/or proliferation.

The use of precursor cells is promising, since, in addition to their plasticity, precursor cell therapy can be used to generate sufficient cell numbers from small tissue samples from individual donors, for the ultimate transplantation back into the donor, thus avoiding the problems of finding a compatible donor and of transplant rejection by the immune system. This technology promises the ability to produce a large number of cells efficiently in culture, and avoids many of the technical and ethical limitations associated with the use of primary embryonic tissue in current transplantation regimes.

Neural precursor cells exist in various regions of the CNS throughout the mammalian lifespan and there has been a growing interest in the characterization of neural precursor cells for basic developmental biology studies, drug screening and for therapeutic applications to the damaged brain. Much research is currently focused on promoting endogenous neural reconstruction and using neural precursor cells in cell replacement or recruitment strategies for the treatment of a variety of human neurological conditions including Parkinson's disease, ALS, Huntington's disease, Alzheimer's, multiple sclerosis and ischemic brain injury. Transplantation of neural precursor cells or their derivatives into a host brain represents a potential treatment for many neurodegenerative diseases, especially Alzheimer's and Parkinson's disease which are characterized by a continuous loss of specific populations of neurons associated with progressive loss of function.

Self-renewing neural precursor cells, with the in vitro capacity to produce all the major cell types of the brain, would be beneficial for transplanting. Since neural precursor cells could give rise to the principal cellular phenotypes comprising the mature CNS, neurons, astrocytes and oligodendrocytes, they may also provide accessible model systems for studying neural development. In addition, because of their migratory behaviour, neural precursor cells also have the potential clinical applications as cellular vectors for gene delivery and the expression of therapeutic proteins.

Additionally, bFGF (basic FGF) has been identified as an essential mitogen for the proliferation of adult and embryonic stem cells derived from several tissues including the central nervous system (reviewed by [1]), bone marrow [2], retina [3], skeletal muscle [4] and very recently, even, adult heart. The recent observation that bFGF-dependent, c-Kit positive multipotent stem cells can be isolated from adult heart (approx. 1 in 1×10⁴ myocytes; [5]), grown as a bFGF-dependent self-renewing population in vitro, and then induced with dexamethasone (in the absence of FGF) to generate cardiomyocytes, smooth muscle cells and endothelial cells, provides convincing evidence that cardiac stem cells may be used to treat cardiac disease [5]. Moreover, injection of these cells into an ischemic rat heart functionally repaired the myocardium in the heart in vivo. The observation that adult skin also contains nestin-positive, EGF and bFGF-dependent multipotent stem cells termed, skin-derived precursor cells (SKPs; [6]), that can be differentiated into multiple cell types in vitro, including neurons, astrocytes and smooth muscle cells (SMCs), holds the potential of establishing self-derived stem cells from skin samples for transplantation and repair if the mechanisms regulating SKP proliferation and differentiation can be further elucidated.

While considerable recent progress has been made in terms of developing new techniques for the long-term culture of human precursor cells, the successful clinical application of these cells is presently limited by an imperfect understanding of precursor cell proliferation. The factors controlling precursor cell survival, undifferentiated state, proliferation, and cell-cycle number are beginning to be identified, but the links between them remain unclear. In order to make precursor cell therapy viable, an effective method must be developed to collect, isolate and grow a plentiful supply of precursor cells that can meet stringent testing requirements for autoimmunity and compatibility.

Culturing long-term precursor cell populations without differentiation has been difficult using current techniques and is limiting practical precursor cell applications. Therefore, a need exists for novel methods to create a large standardized supply of precursor cells suitable for precursor cell therapy.

### SUMMARY OF THE INVENTION

The present invention relates to methods for proliferating precursor cells using FRS3, and the use of these cells and their progeny for therapeutic, research and drug screening purposes.

In one aspect, there is provided a method of proliferating a precursor cell comprising modulating the expression of FRS3 in the precursor cell.

In another aspect, there is provided a method of treating a disorder characterized by the premature death or malfunction of a specific cell type comprising administering to a patient a precursor cell that is the precursor cell for the specific cell type and that has been proliferated by modulating the expression of FRS3 in the precursor cell.

In a further aspect, there is provided a method of treating a disorder characterized by the premature death or malfunction of a specific cell type comprising administering to a patient a precursor cell that is the precursor cell for the specific cell type, the precursor cell comprising a nucleic acid molecule encoding FRS3.

In yet another aspect, there is provided a precursor cell comprising a nucleic acid molecule encoding FRS3.

In yet a further aspect, there is provided a progeny cell of the precursor cell described herein that has been induced to differentiate.

In still yet a further aspect, there is provided a pharmaceutical composition comprising the precursor cell or the progeny cell described herein and a pharmaceutically acceptable carrier.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 depicts the alignment of the amino acid sequence of human FRS3 (hFRS3) [SEQ ID NO.:1] relative to mouse FRS3 (mFRS3) [SEQ ID NO.:2], human FRS2 (hFRS2) [SEQ ID NO.:3] and mouse FRS2 (mFRS2) [SEQ ID NO.:4];

FIG. 2 is A: a schematic diagram of the microinjection construct used to make FRS3 transgenic mice; B: an agarose gel depicting vector transcript expression in E9.0 through E15 FRS3 transgenic animals as determined by RT-PCR; C: Western blots showing FRS3 expression in brain and spinal cord lysates (5 μg total protein) from E10 and E12 hFRS3 positive and negative littermates; and D: photographs of immunohistochemical staining for FRS3 in control and FRS3 animals at E12;

FIG. 3 is A: graphs depicting the physical measurements within the neural tube in E14 negative littermates and FRS3 animals (n=15) at T4 level of the developing spinal cord, the insets showing photographs of thin sectioned neural tubes with the different regions indicated; B: thin section photographs depicting DRG duplications and triplications found in FRS3 transgenics (1I3, 22P3, 23B2), relative to a control animal, at E12 (arrows); C: photographs of longitudinal sections of DRGs from a CD1 and an FRS3 transgenic mouse at P0; and D: photographs of toluidine blue stained E16 dorsal root ganglia showing large neuronal cells (arrows) and smaller diameter cells (arrowheads);

FIG. 4 is graphs depicting A: DRG cell counts across three FRS3 over-expressing lines established from independent microinjections compared to CD1 and negative littermates at E14; and B: DRG cell counts averaged across the three strains between E14 and P0;

FIG. 5 is A: photographs of in situ hybridization for TrkC and TGFβ1 in E10 and E12 dorsal root ganglia showing changes in transcript expression in FRS3 transgenic animals; B: photographs of immunohistochemical staining for FGFR1 and FGFR3 (E14) in control and FRS3 transgenics; C: photographs of sections showing neurogenin 2 expression from E10 through E14 in control and FRS3 transgenic DRG; D: a schematic representation of normal (solid lines) and hFRS3 transgenic (dashed lines) expression levels of neurogenin 2 (Ngn2) and 1 (Ngn1), and E: photographs showing TrkA and TrkB expression changes in dorsal root ganglia of E14 embryos;

FIG. 6 is photographs depicting A: TrkA and TrkB in situ hybridizations in P0 FRS3 animals and control littermates; B: Ki67 immunohistochemistry to detect proliferating cells in the E14 dorsal root ganglion (arrows; positive signal is black); and C: expression of the Wnt receptor, Frizzled2, in E12 control and FRS3 animals;

FIG. 7 is A: Fluorescent in situ hybridization at E10 for sonic hedgehog (Shh) showing ventral neural tube and notochord expression in control littermates and decreased expression in FRS3 animals (circles show areas of Shh expression). Lower panels are increased magnification of upper panels. B: Versican immunohistochemistry in control littermates and FRS3 transgenic animals. Arrows point to locations where versican is reduced in E12-E14 FRS3 animals;

FIG. 8 is A: a graph showing levels of NC cell delamination in FRS3 and control littermate E9 embryos in culture; B: a graph showing levels of NC cell proliferation in FRS3/LacZ and control E9 embryos at 72 hours in culture; C: photographs of agarose gels of PCR products amplified from genomic DNA from FRS3 and control littermate E9 embryos used in above neural crest cultures for actin (positive control and FRS3); and D: photographs of agarose gels of RT-PCR products produced from RNA from embryos used in NC cultures showing actin (left) and FRS3 (right) expression after 72 hours of culture;

FIG. 9 depicts FRS3-induced FGF-dependent in vitro proliferation of neural crest-derived stem cells (NCSCs) after 3 weeks in culture; A: a graph showing total cell counts of NCSCs from control as compared to FRS3 expressing mice; B: a Western blot showing increased expression of transgenic FRS3 at 1 week and 2 weeks in vivo in NCSC's or skin-derived precursor cells (SKPs); C: a control Western blot showing comparable levels of protein (anti-actin) loaded in each lane; and

FIG. 10 is graphs demonstrating the effect of FRS3 on the FGF-dependent in vitro proliferation of SKPs in culture; A: Total cell counts of SKPs derived from control CD1 and nestin-FRS3 expressing transgenic mice; and B: BrdU incorporation (a marker of proliferation) in SKPs derived from control CD1 and FRS3 transgenics.

### DETAILED DESCRIPTION

Many growth factors with stimulatory effects on the proliferation, differentiation and development of precursor cells have been identified. Precursor cell culturing techniques, depending on their origin and cell lineage, typically include one or more growth factors including Fibroblast Growth Factor (FGF), to help control cell proliferation and cell fate. FGF and the neurotrophins (including Nerve Growth Factor (NGF); Brain-derived Growth Factor (BNDF), Neurotrophin 3 (NT-3) and Neurotrophin 4/5 (NT-4/5)) interact with and activate their respective cell surface receptor tyrosine kinases, which in turn stimulate a series of intracellular signaling molecules that regulate, in a coordinated manner, many facets of development, including patterning ([7]; [8], for review see [9]), central nervous system development ([10]; [11]; [12], for review see [9]), and neural crest (NC) migration/proliferation (for review see [9]).

The FGF Receptor Substrate 2 and 3 (interchangeably referred to as FRS2, SNT1 and FRS2α and FGF Receptor Substrate 3 (interchangeably referred to as FRS3, SNT2 and FRS2β) are small adapter proteins that link activated FGF receptors and the Trk family of neurotrophin receptors to downstream signaling molecules. It has been previously shown that FRS2 is required for increased neuritogenesis, neuron survival ([13]; [14]) and differentiation. FRS2 and FRS3 associate with the FGFR1 constitutively in a phosphorylation independent manner, while association with the neurotrophin receptors (Trk receptors) in response to NGF signaling requires receptor phosphorylation for interaction with FRS2 [15]; [14]; [16]; [17]; [18] and with FRS3 [19]. Previous work has shown that FRS2 is required for neuron survival and differentiation [20]; [13]. FRS3 shares similar structure to FRS2, but until now, the role for FRS3 has been unknown.

Developmental studies show that FRS2 and FRS3 are both dynamically expressed during embryo development in the mouse, with FRS3 more spatially restricted than FRS2 [21]. FRS3 is expressed in several adult tissues, and in the embyro almost exclusively in the developing spinal cord, the ventricular layer of the neuroepithelium of the brain, and condensing somites. FRS2 is much more widely spread throughout the embryonic nervous system [21], with expression throughout all layers of the developing neural tube (NT), brain and in dorsal root ganglia (DRG). Spatial and temporal differences in expression of these two adapters point to different roles in embryonic development. The mouse gene knockouts of FRS2 revealed that FRS2 was required for some vital facet of development, without expression embryonic lethality occurred by E7-E7.5, although the reason behind mortality was not determined [22].

The present methods are based on the observation that FRS3, an FGF and neurotrophin receptor signaling protein, can be used to successfully expand a population of precursor cells without differentiation. In addition, the expression of FRS3 within a precursor cell population can induce increased and/or continuous proliferation of precursor cells, resulting in lower culture times and more rapid proliferation rates than normally seen in precursor cell populations. Thus, FRS3 may be used to expand FGF-dependent precursor cell populations in vitro to generate sufficient quantities of differentiating cell types for transplantation and repair of biological deficits/injuries in vivo.

Thus, there is provided a method of proliferating a precursor cell comprising modulating the expression of FRS3 in the precursor cell.

The term “FRS3” as used herein includes homologs of FGF Receptor Substrate 3, and any derivative, variant, or fragment thereof that is capable of inducing proliferation of a precursor cell, or capable of causing a precursor cell to respond to growth factors such as FGF and neurotrophins but not to epidermal growth factor, EGF. A polynucleotide sequence or polypeptide sequence is a “homolog” of, or is “homologous” to, another sequence if the two sequences have substantial identity over a specified region and the functional activity of the sequences is conserved (as used herein, the term ‘homologous’ does not infer evolutionary relatedness). Two polynucleotide sequences or polypeptide sequences are considered to have substantial identity if, when optimally aligned (with gaps permitted), they share at least about 50% sequence identity, or if the sequences share defined functional motifs. In alternative embodiments, optimally aligned sequences may be considered to be substantially identical (i.e. to have substantial identity) if they share at least 60%, 70%, 75%, 80%, 85%, 90%, 95%, 96%, 97%, 98%, 99 identity over a specified region. An “unrelated” or “non-homologous” sequence shares less than 40% identity, though preferably less than about 25% identity, with a polypeptide or polynucleotide of the invention over a specified region of homology. The terms “identity” and “identical” refer to sequence similarity between two peptides or two polynucleotide molecules. Identity can be determined by comparing each position in the aligned sequences. A degree of identity between amino acid sequences is a function of the number of identical or matching amino acids at positions shared by the sequences, i.e. over a specified region. Optimal alignment of sequences for comparisons of identity may be conducted using a variety of algorithms, as are known in the art, including the ClustalW program, available at http://clustalw.genome.ad.jp, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/).

A variant or derivative of FRS3 refers to a fragment, either alone or contained in a fusion or chimeric protein, which retains the ability to induce or enhance proliferation of a precursor cell or to cause a precursor cell to respond to growth factors such as FGF and neurotrophins but not to epidermal growth factor, EGF, or an FRS3 that has been mutated at one or more amino acids, including point, insertion or deletion mutation, but still retains the ability to induce or enhance proliferation of a precursor cell or to cause a precursor cell to respond to growth factors such as FGF and neurotrophins but not to epidermal growth factor, EGF, as well as non-peptides and peptide mimetics which possess the ability to mimic the biological activity of FRS3. A variant or derivative therefore includes deletions, including truncations and fragments; insertions and additions, including tagged polypeptides and fusion proteins; substitutions, for example conservative substitutions, site-directed mutants and allelic variants; and modifications, including peptoids having one or more non-amino acyl groups (q.v., sugar, lipid, etc.) covalently linked to the peptide and post-translational modifications. As used herein, the term “conserved amino acid substitutions” or “conservative substitutions” refers to the substitution of one amino acid for another at a given location in the peptide, where the substitution can be made without substantial loss of the relevant function. In malting such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. Conservative changes can also include the substitution of a chemically derivatised moiety for a non-derivatised residue, for example, by reaction of a functional side group of an amino acid.

Variants and derivatives can be prepared, for example, by substituting, deleting or adding one or more amino acid residues in the amino acid sequence of a native acetylcholine-gated chloride channel or fragment thereof, and screening for biological activity. Preferably, substitutions are made with conservative amino acid residues, i.e., residues having similar physical, biological or chemical properties. A skilled person will understand how to make such derivatives or variants, using standard molecular biology techniques and methods, described for example in Sambrook et al. ((2001) Molecular Cloning: a Laboratory Manual, 3^{rd} ed., Cold Spring Harbour Laboratory Press), and how to test such derivatives or variants for their ability to induce or enhance proliferation of a precursor cell or to cause a precursor cell to respond to growth factors such as FGF and neurotrophins but not to epidermal growth factor, EGF.

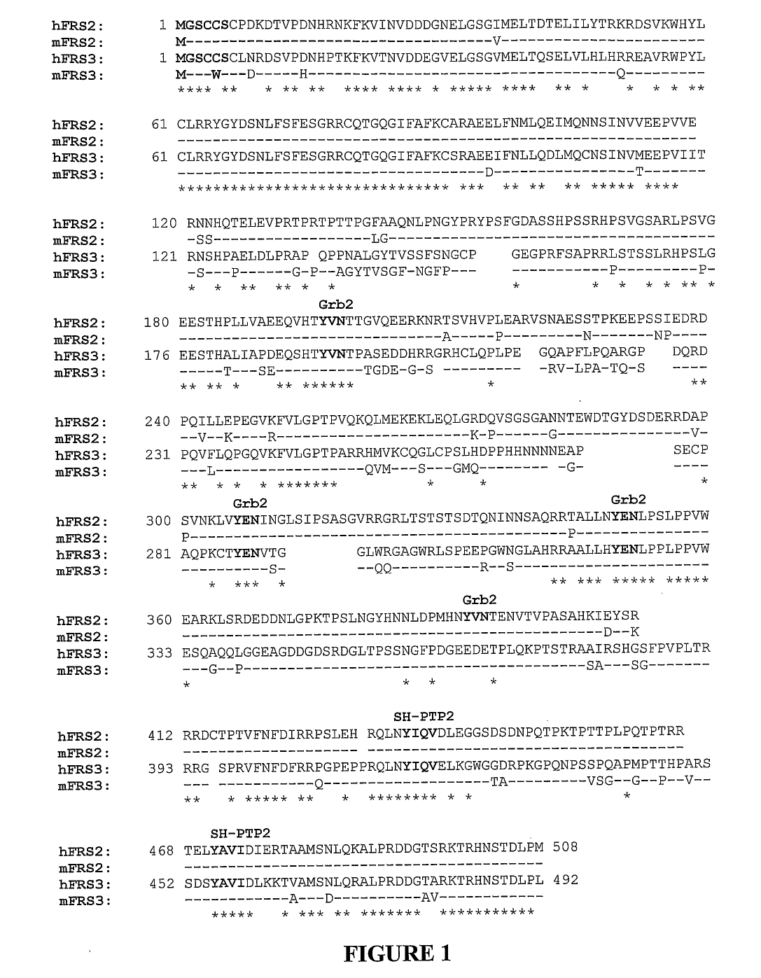

The amino acid sequence of FRS3 homologs from humans [SEQ ID NO.:1] and from mouse [SEQ ID NO.:2] are set out in FIG. 1, and are aligned with the amino acid sequences of FRS2 from human [SEQ ID NO.:3] and mouse [SEQ ID NO.:4]. Gene Bank Accession Numbers for human FRS3 nucleotide and amino acid sequences are NM 006653 and BC010611. In FIG. 1, conserved binding sites for Grb22 (YXN) and SH-PTP2 are shown in bold, dashes indicate conservation of a residue between mouse and human homologs of either FRS2 or FRS3 and asterisks indicate a residue conserved among all four proteins.

A “precursor cell” as used herein refers to a cell that is undifferentiated or partially undifferentiated, and which can divide and proliferate to produce undifferentiated or partially undifferentiated cells or can differentiate to produce one or two differentiated or specialized cells. Precursor cell includes an undifferentiated stem cell that can divide to produce two undifferentiated cells or one undifferentiated cell and one differentiated cell or a partially differentiated precursor cell that can divide to produce two partially differentiated cells or two differentiated cells. Precursor cell as used herein includes precursors cells that are dependent on FGF for proliferation, although such cells may also be responsive to additional growth factors. A precursor cell may be pluripotent, which means that the cell is capable of self-renewal and of trans-differentiation into multiple tissue types upon differentiation.

The precursor cell can be obtained from a variety of sources including, but not limited to, neuronal tissue, peripheral blood, bone marrow, cardiac muscle, liver, retina, skeletal muscle, kidney, pancreatic, spleen, intestinal, lung, skin, umbilical cord cells including umbilical vein endothelial cells, as well as embryonic cells, including embryonic stem cells. Preferably, the precursor cell is derived from adult bone marrow, adult skin or peripheral blood. The precursor cell can be derived from any animal, including a mammal, and particularly from a rodent or a human. The precursor cells used in the methods of the invention, when used to treat a patient, may be patient derived (autologous) or from a donor of the same species (allogeneic) or of a different species (xenogeneic). If the precursor cell is xenogeneic, preferably the cell is a nude cell, which is a cell that has been modified to not express, or to have reduced or minimal expression of, surface antigens that would induce an immune response in the patient being treated.

The term “cell” as used herein refers to and includes a single cell, a plurality of cells or a population of cells, unless otherwise specified.

Proliferating, proliferate, and proliferation, and inducing or enhancing proliferation refer to inducing or stimulating a precursor cell, or enhancing or increasing the ability or rate of a precursor cell, to divide without further differentiation, resulting in an increase in the number of precursor cells. That is, an undifferentiated precursor cell is proliferated when it divides to produce two undifferentiated precursor cells that are the same as the parent cell. Similarly, a partially differentiated precursor cell is proliferated when it divides to produce two partially differentiated precursor cells that are the same as the parent partially undifferentiated precursor cell. Inducing a precursor cell to proliferate means causing a cell that was previously not undergoing division to divide. Enhancing proliferation of a precursor cell means increasing the proliferation rate of a precursor cell already undergoing division, or increasing the response of a precursor cell to growth factors such as FGF or a neurotrophin. Maintaining proliferation of a precursor cell means inducing a proliferating precursor cell to continue to proliferate at least at the same rate.

A precursor cell, for example, obtained from an animal, may be cultured in growth medium, using standard cell cultures that are known in the art, and which medium may include growth factors such as FGF or neurotrophins at a concentration sufficient to cause the precursor cells to proliferate. In one embodiment, concentrations of a given growth factor are in the range of about 25 ng/ml to 50 ng/ml. For example, the precursor cells may be cultured in a plate or dish, or may be grown in batch in a flask or fermentor.

The precursor cell is then treated in order to modulate expression of FRS3 within the cell. FRS3 functions within the cell, and thus the present method relates to introducing FRS3 within a precursor cell or increasing levels of FRS3 within the cell.

Modulating expression includes modulating the expression levels of native FRS3 by chemical or by genetic methods, including by exposure to a chemical or compound that increases expression of the FRS3 or by introducing a nucleic acid molecule or expression cassette encoding a regulatory factor that results in increased expression of FRS3 in the precursor cell.

Modulating the expression of FRS3 in a precursor cell also includes genetically modifying the precursor cell to include a nucleic acid molecule encoding FRS3, including an expression cassette comprising the FRS3 coding region. It will be understood that for the FRS3 to be expressed in the genetically modified precursor cell, the nucleic acid molecule will contain the coding region of FRS3 operably linked to the necessary regulatory regions required to effect expression, including a suitable native or heterologous promoter region and enhancer elements. The precursor cells may already express FRS3, and therefore the nucleic acid molecule encoding FRS3 may be designed to express FRS3 at levels above the natural levels of expression in the precursor cell. Alternatively, the precursor cell may not express FRS3 and is in the present method genetically modified to produce FRS3, at levels sufficient to cause the precursor cell to proliferate.

Genetic modification can be achieved using molecular biology and cloning methods known in the art. For example, the precursor cell may be transformed or transfected with a vector designed to express FRS3 from a native or heterologous promoter, and the promoter may be a constitutive, transient or inducible promoter, and may direct expression at basal or heightened levels of expression. Suitable vectors include bacterial plasmids or viral vectors including viral genomes. For example, a retroviral, a lentiviral or an Adenoviral vector may be used.

The genetically modified precursor cell may be cultured in vitro, as described above, under suitable conditions to express the FRS3 gene in the cell and to allow the cell to proliferate in culture. Where an inducible promoter is used, the culturing will include the compound or growth conditions necessary to induce expression of the FRS3 gene.

The precursor cell may also be genetically modified to include one or more nucleic acid molecules encoding a therapeutic protein or peptide, including a nucleic acid molecule comprising a therapeutic transgene. It will be understood that the nucleic acid will comprise the coding region for a therapeutic protein or peptide as well as necessary regulator regions required to effect expression of the therapeutic protein or peptide in the cell. Such regulatory regions include a suitable promoter region, such as a native or heterologous promoter, as well as enhancer elements. For example, a vector, including a vector used to express FRS3 in the precursor cell, may be designed to express a therapeutic transgene or a therapeutic protein or peptide, particularly if the precursor cell is to be used to treat a disease or disorder in a patient.

The term “therapeutic gene” or “therapeutic transgenes” as used herein is intended to describe broadly any gene the expression of which effects a desired result, for example, a gene involved in cell differentiation, or a gene involved in the treatment of diabetes, including insulin secretion or uptake, or treatment of cardiac disorders. A therapeutic protein or peptide is a protein or peptide, that when expressed in the precursor cell, has a therapeutic effect on the cell, or which effects a desired result within the precursor cell.

Thus, FRS3 may be used to create an in vitro population of undifferentiated or partially differentiated precursor cells, useful for various in vitro purposes, including studying the differentiation pathways and mechanisms of certain cell types and the effects of certain compounds, cell factors or proteins on the differentiation or proliferation of precursor cells, studying the ability of precursor cells to produce and/or secrete growth factors, hormones or extracellular molecules that may themselves regulate the proliferation or differentiation of precursor cells or other partially differentiated cell types, and also including drug screening methods in certain populations of undifferentiated, differentiated or partially differentiated cell types.

Once a desired level of proliferation is achieved, a population of precursor cells can be used to create a population of progeny cells for in vitro use or for in vivo therapeutic use. The amount of proliferation can be measured by cell counts and by talking a subset of cells and assaying for the incorporation of the thymidine analogue, BrdU.

Thus, the above method can further include inducing the precursor cell to differentiate after proliferation to produce progeny cells. Inducing differentiation is accomplished by exposing the precursor cell to an additional cellular factor, such as particular growth factors, by withdrawal of serum or growth factors, such as FGF, and/or by the addition of various pharmacological reagents, such as dexamethasone enzyme inhibitors and other compounds which can cause the cell to differentiate.

A progeny cell is a cell derived from a precursor cell which has undergone differentiation or partial differentiation, and which is more differentiated than the original precursor cell. For example, a precursor cell that is a brain stem cell may differentiate to produce astrocytes, oligodendrocytes or neurons.

The precursor cells can be tested to determine if they underwent differentiation by testing for the presence or absence of precursor cell markers. For example, neural precursor cells express the Nestin and Frizzled markers and should not express melanin, C-kit, GFAP, smooth muscle actin or neurofilament 160. In another example, undifferentiated cardiac precursor cells are Lin⁻ and C-kit⁺.

Thus, there is presently provided a precursor cell, or a progeny cell differentiated from the precursor cell, which comprises a nucleic acid molecule encoding FRS3, and which is therefore genetically modified to express FRS3. The precursor cell or the progeny cell differentiated from the precursor cell may further comprise one or more therapeutic transgenes or a nucleic acid molecule encoding one or more therapeutic proteins or peptides.

The production of a population of precursor cells is useful for precursor cell therapy in which precursor cells, or progeny cells derived from precursor cells, are provided to a patient to replace or compensate for cells which have died or which do not function properly, thereby treating a disease or disorder. Thus, there is also presently provided a method of treating a disorder characterized by the premature death or malfunction of a specific cell type comprising administering a precursor cell that is a precursor cell for the specific cell type and which is proliferated by the above-described method, or a progeny cell differentiated from such a precursor cell, to a patient. The cell may be a precursor cell or a progeny cell that is genetically modified to express FRS3, and optionally further genetically modified to express one or more therapeutic transgenes or therapeutic proteins or peptides.

A “disorder characterized by the premature death or malfunction of a specific cell type” refers to a disease or disorder in which a specific cell type in an individual has prematurely died, or which no longer, or never did, function at sufficient level so as to prevent the development of the disease or disorder. In a healthy individual, such a specific cell type would be alive, including being in a quiescent or senescent state, and would function at a level which does not cause disease or a disorder. The disorder includes, but is not limited to, cancer including leukemia, neurological diseases such as Parkinson's Disease, Alzheimer's and ALS (Lou Gehrig's Disease), CNS damage including spinal cord injury and Multiple Sclerosis, cardiac damage, liver damage, kidney damage, pancreatic damage, retinal damage, intestinal damage, skeletal muscle damage including Muscular Dystrophy, lung damage and diabetes.

Treating a disease or disorder refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). “Treating” can also mean prolonging survival of a patient beyond that expected in the absence of treatment. “Treating” can also mean inhibiting the progression of disease, slowing the progression of disease temporarily, although more preferably, it involves halting the progression of the disease permanently.

The patient is any patient suffering from a disorder characterized by the premature death or malfunction of a specific cell type and who is in need of such treatment. The patient may be any animal, including a mammal, particularly a human.

The precursor or progeny cells are administered to the patient by delivery to the site of the specific cell type which has prematurely died or which has malfunctioned, using methods known in the art, including by surgical implantation or by injection, for example at the site of a tissue or organ, such as the liver, pancreas, cardiac tissue, brain or spinal cord. For example, if the disorder being treated is diabetes, the precursor or progeny cells may be cells that can differentiate to become D islet cells, and may be implanted or injected in an islet in the pancreas. In another example, if the disorder being treated is cardiac damage, a precursor cell that can differentiate to become a cardiomyocyte may be implanted or injected into heart muscle. Similarly, in other examples precursor cells can be differentiated into cells specific for lung, kidney, liver, intestinal wall, retinal or skeletal muscle tissue.

An effective amount of precursor cells or progeny cells are administered to the patient. The term “effective amount” as used herein means an amount effective, at dosages and for periods of time necessary to achieve the desired result, for example, to treat the specific disorder.

The number of total precursor or progeny cells to be administered will vary, depending on the disorder or disease to be treated, the type of cell that is administered, the mode of administration, and the age and health of the patient.

To aid in administration, a precursor cell, or a progeny cell differentiated from the precursor cell, which comprises a nucleic acid molecule encoding FSR3, may be formulated as an ingredient in a pharmaceutical composition. Therefore, in a further embodiment, there is provided a pharmaceutical composition comprising a precursor cell, or a progeny cell differentiated from the precursor cell, which comprises a nucleic acid molecule encoding FRS3, and a pharmaceutically acceptable diluent. The invention in one aspect therefore also includes such pharmaceutical compositions for use in treating a disorder characterized by the premature death or malfunction of a specific cell type. The compositions may routinely contain pharmaceutically acceptable concentrations of salt, buffering agents, preservatives and various compatible carriers. For all forms of delivery, the precursor cell or progeny cell may be formulated in a physiological salt solution.

The pharmaceutical compositions may additionally contain other therapeutic agents useful for treating the particular disorder. Alternatively, the pharmaceutical composition may contain growth factors or cellular factors that facilitate cell survival and induce proliferation or differentiation of the precursor cell or the progeny cell when delivered to the site of the disorder.

The proportion and identity of the pharmaceutically acceptable diluent is determined by chosen route of administration, compatibility with live cells, and standard pharmaceutical practice. Generally, the pharmaceutical composition will be formulated with components that will not kill or significantly impair the biological properties of the live precursor or progeny cells.

The pharmaceutical composition can be prepared by known methods for the preparation of pharmaceutically acceptable compositions suitable for administration to patients, such that an effective quantity of the precursor cells or progeny cells, and any additional active substance or substances, is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the precursor cells or progeny cells, in association with one or more pharmaceutically acceptable vehicles or diluents, and contained in buffer solutions with a suitable pH and iso-osmotic with physiological fluids.

The pharmaceutical composition may be administered to a patient in a variety of forms depending on the selected route of administration, as will be understood by those skilled in the art. The composition of the invention may be administered surgically or by injection to the desired site.

Solutions of the precursor cells or the progeny cells may be prepared in a physiologically suitable buffer. Under ordinary conditions of storage and use, these preparations contain a preservative to prevent the growth of microorganisms, and that will maintain the live state of the cells. A person skilled in the art would know how to prepare suitable formulations. Conventional procedures and ingredients for the selection and preparation of suitable formulations are described, for example, in Remington's Pharmaceutical Sciences and in The United States Pharmacopeia: The National Formulary (USP 24 NF19) published in 1999.

In different embodiments, the composition is administered by injection (subcutaneously, intravenously, intramuscularly, etc.) directly at the desired site where the cells that have prematurely died or are non-functional are located in the patient.

The dose of the pharmaceutical composition that is to be used depends on the particular condition being treated, the severity of the condition, the individual patient parameters including age, physical condition, size and weight, the duration of the treatment, the nature of concurrent therapy (if any), the specific route of administration and other similar factors that are within the knowledge and expertise of the health practitioner. These factors are known to those of skill in the art and can be addressed with minimal routine experimentation.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

### EXAMPLES

### Summary

Here we report spinal NT and DRG phenotypes resulting from transgene over-expression of human FRS3 (hFRS3) in mice under the second intronic region enhancer of the nestin gene and minimal TK promoter. These mice show gross abnormalities in the DRG, including duplications and triplications in structures that form in more ventral locations to the normally located DRG. There is a statistical increase in the number of cells found in both normal and duplicated DRG beginning at E10 and persisting to E16. By P0, most duplicated DRG structures exhibit cell loss when compared with control DRG unless directly in contact with nerve tracts, in which case cell numbers remain abnormally high. Ganglia that are formed in the normal location in FRS3 over-expressing animals show a marked increase in cell number over control DRG. We also show a reduction of sonic hedgehog (Shh) expression in the NT which correlates with the formation of ectopic DRG structures, providing evidence of a reciprocal interaction between FGF and Shh signaling and patterning systems.

Materials and Methods

Transgenic mice: Full length human FRS3 cDNA was derived from amplification of a human cDNA library (Clontech), subcloned into pcDNA3.1-myc-his first, then excised and subcloned into pIRES2-EGFP. NcoI digest produced a fragment containing fall length FRS3, myc tagged with IRES on the 3′ end, which then was subcloned into Nestin-TK (gifted from Dr. McMahon, Harvard University) to give the final microinjection cassette. Transgenic mice were derived by pronuclear injection of linearized Nestin-TK-hFRS3-LacZ constructs into CD1 mice (Robarts Barrier Facility, Robarts Research Institute, London, Ontario, Canada). Animals were assayed for the presence of human FRS3 by genomic PCR from tail sample, and RT-PCR of embryonic brain RNA with primers specific to the human gene sequence DNA (primers: forward: AGC CAC CCA ATG CTC TAG [SEQ ID NO.:5]; reverse: GTG GGG GCA GGT TCT CAT AGT GCG [SEQ ID NO.:6]). Of 9 lines established, 3 were found to be germline transmitting and expressed hFRS3 when examined by RT-PCR. These lines were maintained for study. Each line was established from a single founder animal, and all lines have been maintained as heterozygous animals. No homozygotic animals were found on any lines, as tested by matings with CD1 animals and assaying all embryos for the presence of the gene. Homozygotic animals were determined to be lethal at approximately E9-E9.5 (to be published elsewhere). Litters were tested from each breeding pair to confirm germline transmission and RNA expression. All animals carried to term were screened for hFRS3 genotype by tail sampling and amplification with hFRS3 specific primers (to be published elsewhere). All data herein are presented from heterozygous animals.

Animals were also produced by microinjection of the above cassette without the hFRS3 coding region to produce Nestin-TK-LacZ lines. These animals were bred to homozygousity, and cross-bred to heterozygous hFRS3 transgenic mice to supplement the inactive LacZ coding found upon sequencing of the hFRS3 lines for neural crest experiments.

Ovulation, implantation sites and embryo loss: Female transgenic animals were mated with either CD1 or transgenic males from the same line. Two days after vaginal plug females were euthanized by carbon dioxide inhalation, and ovaries, oviducts and uterii dissected. Ovulation sites were counted on the surfaces of both ovaries. Embryos were flushed from oviducts and upper uterine horns with warmed PBS, counted and compared with number of ovulations. For determination of implantation sites, ovaries and uterii were removed from plugged females at E7, focal uterine enlargements counted and dissected to confirm embryo presence and compared to the number of corpora lutea present on both ovaries.

Transgene expression analyses: Total RNA was extracted from E12 or E14 embryo heads by TRIzol (Invitrogen) as per manufacturer's protocol. Poly(A) RNA was digested for 1 hour with 1 unit RNAse-free DNAse (Invitrogen), re-extracted with phenol:chloroform:isoamyl alcohol and reverse transcribed using Superscript RT (Invitrogen) as per manufacturer's protocols. Amplification of the resulting cDNA was performed as described above with primers for hFRS3, actin, β-galactosidase or mFRS3.

X-gal staining: Embryos were examined for X-gal staining as follows: Embryos were dissected from maternal membranes and fixed for 1 hour in 4% paraformaldehyde pH 7.2. Embryos were then rinsed 3 times for 15 minutes each in rinse buffer (5 mM EGTA, 0.01% deoxycholate, 0.02% NP40, 2 mM MgCl2), then incubated in stain solution (rinse buffer plus 5 mM K4Fe(CN)6, 5 mM K3Fe(CN)6 and 1 mg/ml X-gal) for 12-15 hours at 37° C. Stained embryos were then rinsed in PBS, dehydrated, embedded in paraffin wax and sectioned at 5 um thickness in the transverse plane. Sections were dewaxed to PBS, counterstained with eosin, washed twice in 70% ethanol, rinsed in PBS, coverslipped with aqueous mounting medium (Geltol; Immunon) and photographed with an Olympus microscope under normal light.

In situ hybridization: In situ hybridization was performed as described in [21] for neurogenin 1, neurogenin 2 (Ngn1, Ngn2; constructs were generous gifts from Dr. D. J. Anderson, Howard Hughes Medical Institute, Caltech, Calif., USA), Sox10, Pax3, bone morphogenic protein 2 (BMP2), BMP4, Shh, FGFR1, TrkA, TrkB, TrkC, transforming growth factor beta-1 (TGFβ1), Snail, and FRS3 (250 bp coding region fragment of FRS3). Fragments for Sox10, BMP2, BMP4, FGFR1, TrkA, TrkB, TrkC, Shh and TGFBβ1 were derived from PCR of cDNA from E10-E16 embryos, Snail and Pax3 fragments were amplified from an E8 mouse cDNA library (Clontech). Amplicons were TA-cloned into pGEMT-Easy (Promega), confirmed by sequencing for both sequence integrity and orientation of insertion and probes in vitro transcribed using SP6 or T7 RNA Polymerase (MBI Fermentas) as per manufacturer's specifications. All riboprobes were fluorochrome tagged with either Texas Red-UTP (Amersham) or Fluorescein-UTP (Amersham).

Embryos from E9 to E14 were dissected free from maternal membranes, and one forelimb removed for DNA analysis by PCR. The remainder of the embryo was fixed in 4% paraformaldehyde for 1 hour at 4° C. Embryos were then dehydrated to 95% ethanol, and paraffin embedded using a Leica Tissue Processor. Embryos were serial sectioned at 5 um thickness in the transverse plane. Sections were then subjected to in situ hybridization as described in [21].

Histology: Embryos between E9 and E16 were dissected from maternal membranes, fixed, embedded and sectioned as above. For P0 pups (day of birth) sections of the spinal cord were cut between T1 and T5, fixed, embedded and sectioned as for embryos. Slides were stained with either Hematoxylin and Eosin (Fisher Scientific), or toluidine blue (Fisher Scientific). Entire neuronal phenotype populations of DRGs were counted from at least 5 positive over-expressing animals from 3 lines of FRS3 animals and from 5 CD1 animals. Counts were performed on all DRG between T2 and T4 of the spinal cord. Counts of all DRG structures were kept separate for each DRG structure, even if several DRG structures were apparent at the same vertebral level in FRS3 over-expressing animals. All cells with a visible nucleus in every section were counted, and all sections in each DRG were counted. Counts were adjusted as per the method of Abercrombie [25] to reduce sampling error by counting cells with nuclei in adjacent sections. Analysis of valiance statistical test was performed using InStat software to determine significance.

Hematoxylin and eosin staining was performed at room temperature. Slides were dewaxed to PBS, stained in Weigert's Hematoxylin (Fisher Scientific) for 5 minutes, rinsed 10 minutes in running water, dipped 5 times in Eosin (Fisher Scientific), cleared in 95% ethanol and coverslipped with permount.

Toluidine blue staining was performed at room temperature. Slides were dewaxed to PBS, dipped 3 times in 1% toluidine blue (in PBS), washed 5 minutes in running water, and coverslipped with Geltol (Immunon).

Ki67, Versican and Frizzled immunohistochemistry: All steps were performed at room temperature until otherwise stated. For Ki67 immunohistochemistry: Sections were dewaxed to 95% ethanol and digested for 10 minutes in 3% hydrogen peroxide in methanol. Sections were then washed twice in water, and blocked for 1 hour in 5% goat serum in PBS. A 1:50 dilution of Ki67 antibody (BD Biosciences) in 5% goat serum was incubated on cells overnight at 4° C. Slides were washed twice in PBS, 1:50 dilution of biotylinated IgG secondary antibody (in 5% goat serum) incubated for 30 minutes, washed twice in PBS and incubated by 1:50 dilution of streptavidin-HRP (in 5% goat serum) for 30 minutes. After washing in PBS, DAB staining was performed with a Vectastain kit (BD Biosciences) as per manufacturer's protocol. Slides were washed in water, counterstained in hematoxylin, dehydrated to xylenes and mounted with permount (Sigma). Sections were photographed with an Olympus microscope with Hoffmann-contrast and a green daylight filter to enhance contrast between the DAB precipitate and tissues (precipitate appears dark brown-black while tissues appear blue-green). For versican immunohistochemistry: Slides were dewaxed and rehydrated to PBS then digested for 30 minutes at 37° C. with 0.5 units chondroitinase (Sigma, resuspended 0.5 units/ml in 0.02% bovine serum albumin fraction V, 50 mM Tris pH 8.0 and 60 mM sodium acetate), washed in PBS and blocked for 30 minutes in 5% goat serum at room temperature. Rabbit anti-versican (Chemicon International) was diluted to 10 ug/ml in blocking buffer and applied to slides overnight at 4° C. Slides were washed twice in PBS, then incubated for 1 hour in horseradish peroxidase conjugated goat-anti-rabbit secondary antibody for 1 hour at room temperature. Following two PBS washes, DAB staining was performed with a Vectastain kit, and slides coverslipped as above.

The anti-Frizzled antibody was from Santa Cruz Inc. Paraffin sections of 5 um thicknesses were obtained from E10 through E14 mouse embryos, both negative littermates and positive hFRS3 embryos (confirmed by PCR of limb bud DNA as to be described elsewhere). Sections were de-waxed to PBS and immunohistochemistry performed as follows: Dewaxed and rehydrated sections were blocked for 30 minutes in 5% goat serum in PBS. Primary antibody (rabbit anti-Frizzled) was used at 10 ug/ml in blocking buffer overnight at room temperature. Sections were washed in PBS twice for 10 minutes each at room temperature. Secondary antibody (Goat anti-rabbit-horseradish peroxidase conjugate) was incubated on slides in blocking buffer for 1 hour at room temperature. Slides were washed twice in PBS and subjected to the DAB colour reaction as above.

Immunocytochemistry on Neural Crest Cultures: Neural crest cultures were examined for GFAP, melanin, Neurofilament 160 and smooth muscle actin before differentiation and post-differentiation by immunocytochemistry. Cultures were prepared from both controls and confirmed FRS3 transgenic animals. Immunocytochemistry was performed blinded to genotyping results to prevent data biasing. Cultures were fixed for 20 minutes in 4% paraformaldehyde at room temperature, washed twice with phosphate buffered saline pH 7.2, and blocked in 10% goat serum in PBS containing 0.1% triton X-100 for 1 hour at room temperature. Primary antibodies were mouse anti-neurofilament 160 (1:500 dilution; Sigma), mouse anti-smooth muscle actin (Sigma; 1:500 dilution), or rabbit anti-glial fibrillary acidic protein (GFAP; 1:100 dilution; Sigma). Secondary antibodies were goat anti-mouse-Cy3; goat anti-mouse RFP or goat anti-rabbit-Cy3 depending on the combination of primary antibodies used. Cultures were examined using 2 primary antibodies, positive cells counted and then the third and final antibody applied and results determined by subtractive analysis between the first and second counts. Melanocytes were identified based on the presence of dark brown-black melanin within the cells and a highly migratory behaviour. Cells that did not contain melanin and did not label with any antibodies were not included in the analysis. Primary antibodies were added to blocking buffer to the dilutions required, and incubated for 30 minutes at room temperature. Cultures were then washed twice for 15 minutes each with PBS containing 0.1% triton X-100 at room temperature, blocking buffer added and secondary antibodies added for 30 minutes at room temperature. Cultures were washed 3 times for 20 minutes each in PBS containing 0.1% triton X-100, coverslipped with Geltol medium (Immunon) and visualized under fluorescence using an Olympus microscope with a mercury lamp immediately. Preps from 4 FRS3 transgenic and 4 negative animals were analyzed.

Neural Crest Cultures: Fibronectin for coating wells was prepared by resuspending lyophilized human fibronectin (Sigma) in sterile Dulbecco's phosphate buffered saline (PBS) to 250 ug/ml and used to coat plates by pipetting into wells and immediately withdrawing excess. Culture medium was applied directly to fibronectin coated wells. Neural crest medium was prepared as follows: 500 ml L-15 with glutamine (Wisent) was combined with 0.027 g imidazole, 2.25 ml Stable Vitamin Mix (defined below), 1.08 g sodium bicarbonate and pH adjusted to 7.35-7.40. Medium was then preconditioned in CO₂ atmosphere. To 463 ml of the above mixture the following were added and then filtered: 500 mg Fraction V bovine serum albumin, 500 ul 1 mM dexamethasone, 5 ml fresh vitamin mix (defined below), 1.8 ml glycerol, 500 ul 5 mg/ml insulin, 5 ml 7 mix (defined below), 13.3 ml DPS mix (defined below), 10 ul 1 mM progesterone, 1 ml 8 mg/ml putrescein, 150 ul 0.1 mM selenious acid, 1 ml 50 mg/ml transferrin, 1 ml 50 mg/ml vitamin E, 80 ul 25 ug/ml basic FGF, 500 ul 0.1 mg/ml EGF and 500 ul 20 ug/ml NGF. Stable vitamin mix was a combination of 0.6 g each of aspartic acid (Sigma), L-glutamic acid (Sigma), L-proline (Sigma), and L-cystine (Sigma); 0.2 g each of p-aminobenzoic acid and beta-alanine (both from Sigma); 80 mg vitamin B-12 (Sigma), 0.4 g each of I-inositol, choline chloride and d-biotin (all from Sigma), 1 g fumaric acid (Sigma), 6 mg coenzyme A, and 100 mg DL-6,8-thiotic acid (Sigma) all mixed in 200 ml water and aliquotted at −80° C. Fresh vitamin mix was 1 mg DMPH4 (Calbiochem), 5 mg Glutathione and 100 mg ascorbic acid in 20 ml water, pH adjusted to 5 with KOH and then stored at −20° C. The 7 mix was 0.063 mg/ml DL-beta-hydroxybutyrate, 0.025 ug/ml cobalt chloride, 0.01 ug/ml oleic acid, 0.1 ug/ml alpha melanocyte stimulating hormone, 0.01 ug/ml prostaglandin E1 and 0.0675 ug/ml T3 (all from Sigma) in L15 medium, stored at −80° C. DPS was 60 g dextrose (Bioshop), 100 ml 200 mM glutamine (Sigma) and 100 ml penicillin/streptomycin (10,000 u/ml penicillin, 10,000 ug/ml streptomycin; Wisent) in 400 ml final volume and filtered prior to use.

Heterozygous Nestin-hFRS3 transgenic females were either mated with homozygotic Nestin-LacZ transgenic males or heterozygous Nestin-hFRS3 transgenic males. The later breeding would generate litters of 25% homozygotic (lethal), 50% heterozygotic and 25% wild-type (non-transgenic for hFRS3) animals. The date of plug was established and designated E0.5. Pregnant adult females were euthanized by CO₂ inhalation at E9.0, and uteri dissected into warmed phosphate buffered saline. Uteri were washed in PBS twice, and embryos dissected using a dissecting scope (Zeiss) into warmed Hank's Balanced Salt Solution (HBSS) using watchmaker's forceps to remove the embryo from the uterus. For neural tube dissections, embryos were rinsed in HBSS and the neural tube was removed from embryos by gentle dissection from other tissues, including somites, using the points of two 22-guage needles, and placed directly into fibronectin-coated 4 well dishes (Nunc) containing 250 ul neural crest medium. Tubes were pressed firmly into the fibronectin coating using the points of the needles, and cultured overnight in a 37° C. incubator in 5% CO₂ in humidified air atmosphere. All remaining tissues from the dissection were placed in 1.5 ml microcentrifuge tubes and used for genotyping. The following day, 10-12 hours post-plating of the tube, tubes were removed with ethanol disinfected watch maker's forceps using a dissecting scope to confirm removal of the entire tube. Neural crest cells remained attached to the fibronectin coating and if partial remains of the tube persisted cultures were washed gently with an additional 250 ul of neural crest medium to remove tube cells (which remain in suspension while crest cells adhere to the fibronectin). Cultures were maintained for 3 weeks in the above atmosphere and culture conditions. Every 48 hours the medium was changed by aspirating the old medium with a pipettor (not vacuum) and replacing it gently with 500 ul neural crest medium pipetted down the wall of the culture well to prevent stripping the fibronectin from the plate. Alternatively, neural crest cells were digested with 0.1% trypsin for approximately 5 minutes and triturated from the plate, and replated at 100 cells per well on fibronectin and followed in culture for 3 weeks with cell counts performed at 1 and 3 weeks to assess proliferation. Counts were performed blinded, with results correlated with the genotyping after completion of counts. A total of 5 litters were examined, consisting of 42 neural tube cultures, with 8 of these being confirmed negative and 34 confirmed FRS3 transgenic. Counts were analysed statistically using ANOVA.

Genotyping: Tissues that were not plated for neural crest preps or for SKP cultures were saved into 1.5 ml microcentrifuge tubes for DNA extraction. Tissues were added to 500 ul tail buffer (0.05M Tris pH8.0, 0.1M EDTA pH8.0, 0.5% SDS) and 0.25 ug proteinase K and incubated overnight at 55° C. Samples were vortexed vigorously after 12-15 hours incubation, 500 ul phenol:chloroform:isoamyl alcohol (25:24:1) added and vortexed again, then spun at 13200 RPM for 10 minutes to separate phases. The aqueous phase was retained to a new tube and re-extracted with phenol:chloroform:isoamyl alcohol as described above. The aqueous phase was retained to a new tube and DNA precipitated with 50 ul 3M sodium acetate (pH6.0) and 500 ul absolute ethanol. Samples were mixed thoroughly by vigorous vortexing, and spun at 13,200 RPM to pellet DNA. Pellets were washed in 500 ul 70% ethanol, respun to pellet, and then dried for 10 minutes in air at room temperature. Pellets were then resuspended in 200 ul sterile water for subsequent PCR amplification of DNA. All animals used in any experiments were genotyped at the time of the experiment.

PCR amplification for genotyping: DNA extracted as above was amplified with Platinum Taq polymerase (Invitrogen) using primers specific for human FRS3 (forward primer: AGC CAC CCA ATG CTC TAG GCT ACA [SEQ ID NO.:7]; reverse primer: GTG GGG GCA GGT TCT CAT AGT GCA [SEQ ID NO.:8]) for 32 cycles with denaturation of 94° C. for 30 seconds, annealing of 58° C. for 30 seconds, and extension of 72° C. for 1 minute. Amplicons were run on a 1% agarose gel stained with ethidium bromide for visualization using UV light.

SKP Cultures: Skin stem cells were derived from FRS3 transgenic or CD1 control mice at either P0 (day of birth) or adults over 2 months of age. Mice were euthanized by CO₂ inhalation, and the skin of the abdomen and flanks wetted with ethanol. Skin patches approximately 2 cm by 2 cm were dissected from the abdomen and flanks of mice, and macerated into as small pieces as possible (approximately 1 mm×1 mm) using small dissecting scissors into warmed Hank's Balanced Salt Solution (HBSS). Skin pieces were washed 3 times in warmed HBSS and then transferred to HBSS containing 0.01% trypsin and 0.05% collagenase for 40 minutes at 37° Celsius with periodic vortexing to aid in dissociation. Tissues were dissociated by pipetting through a flamed glass pasteur pipette several times in HBSS containing trypsin and collagenase, and then 5 ml of culture medium without serum (contents: DMEM-F12 (3:1), 100 ug Fungizone (Invitrogen), 1% pen/strep (Wisent), 20 ng/ml EGF and 40 ng/ml βFGF (Harlan) and 0.1% B27 Supplement (Invitrogen)) was added and vortexed gently. Tubes were spun at 1000 rpm for 5 minutes to pellet cells. Pellets were washed in culture medium without serum 3 times and then resuspended in 1 ml culture medium with serum (contents: DMEM-F12 (3:1), 100 ug Fungizone, 1% pen/strep, 10% rat serum (Wisent) and 0.1% B27 Supplement), poured through a 100 um cell strainer into 10 ml suspension culture flasks (Falcon) and cultured in 10 ml culture medium with serum. Cells were passaged every 7 days by spinning cells down at 1000 rpm for 5 minutes at room temperature, aspirating the supernatant and resuspending in 10 ml new medium. For counts, cells were counted using a hemocytometer, plated at 100 cells per well, and all cells counted every week for 5 weeks to assess increase in cell numbers. SKP cultures were done from 10 adults (5 controls and 5 FRS3 heterozygous adults) and from 30 P0 pups, of which 14 were negative (controls) and 16 were confirmed FRS3 transgenic. Results from adult and P0 SKP cultures were similar. For counts up to 5 weeks, parent cultures were seeded at 100 cells per well in a 6-well dish, and the rest of the preps plated into flasks. All cells were counted at 1 week in the 6-well dishes, while at week 2, 3 and 4 new 6-well dishes were plated from the parent flasks. Separate 6-well dishes were plated from passaged week 1, 2, 3 and 4 cultures at 100 cells per well to assess proliferative capacity of longer term cultures. Results from both these plating methods were similar. Data were analysed by ANOVA.

BrdU Labelling of SKPs: SKPs were isolated from culture medium by centrifugation at 1000 rpm for 5 minutes, resuspended in BrdU labeling medium diluted in SKP culture medium, labelled and BrdU detected by manufacturer's protocol for labelling suspension cells with the BrdU labeling and detection Kit I by Roche Applied Science. SKPs derived from 6 adults (3 control and 3 FRS3 transgenic) and 12 P0 pups (5 controls and 7 FRS3 transgenic) were examined using BrdU incorporation, with similar results for both.

Western Blots: SKP cells or neural crest stem cells were lysed in 50 ul NP40 lysis buffer as described in Meakin et al. (1999). Whole cell lysate protein concentration was determined by protein assay using the Biorad DC protein assay kit as per manufacturer's protocol, and 50 ug of whole cell lysate run on a 10% denaturing PAGE. Antibodies used in probing were affinity purified rabbit anti-FRS3 peptide specific (1:10,000 dilution of 200 μg/ml, directed against the peptide PDGEEDETPLQKPTS [residues 361 to 375 from SEQ ID NO.:1]; GeneMed Synthesis Inc.) and mouse anti-actin (Sigma; 1:10,000 dilution) primary antibodies, goat anti-mouse (1:10,000 dilution) or goat anti-rabbit (1:10,000 dilution) horseradish peroxidase conjugated secondary antibodies and epichemiluminescence (Western Lightning ECL Kit; Perkin Elmer) as per manufacturer's protocol with exposure to x-ray film. Positive controls for FRS3 expression was whole cell lysate from 293T cells transfected with pcDNA3.1-hFRS3-myc-his.

Adenovirus Infection of SKPs: Adenovirus was made bearing the EGFP marker and hFRS3 in the pAD-Easy system previously in our lab. Titers were determined prior to use from previous experiments. As this virus was made in 2001 a 100× drop in infectivity was estimated based on other experiments on-going with similar viruses made at the same time. MOIs of 25, 50, 75, 100 and 150 were tested in one experiment using SKPs from control animals. This experiment is an N=1 only, preliminary data are present from MOI of 25, 50 and 75, as MOIs of 100 and 150 were toxic to the SKPs and resulted in 100% cell death over 3 days.

Control SKPs were plated at 10,000 cells per well for this experiment. Infection was with adenovirus containing hFRS3-EGFP or β-gal-EGFP (control). Using the same control SKP culture as parent culture, 5 wells were plated for each treatment, one for each MOI (25, 50, 75, 100, 150) for a total of 10 wells plated. MOIs were determined and the resultant volume of adenovirus stock from −80° C. storage was thawed and applied directly into SKP culture medium (with serum; SKPs cultured as above). SKPs were allowed to be infected for 48 hours, at which time they were aspirated to 15 ml tubes, spun at 1000 rpm for 5 minutes, washed twice in 5 ml phosphate buffered saline to remove any viruses left, and replated in 3.5 ml culture medium (with serum) in 6 well plates (Sarstedt). All cells expressing EGFP in each well were counted at plating and again 72 hours later to assess proliferation.

Results

Construct expression: The transgene construct was designed to target neural tissue using the second intronic region enhancer of the Nestin gene, and under the minimal TK promoter in transgenic CD1 mice. The reporter β-galactosidase (β-gal) gene was included with an upstream internal ribosomal entry signal for tracking gene expression. Lothian and Lendahl [23] found that β-gal expression driven by a similar system involving the Nestin second intronic region enhancer was observed from E10.5 through E14 in neural progenitors in the central nervous system and earlier in NC. [24] (1999) found that the second intronic region enhancer targeted expression of transgenes to the central nervous system, specifically to neural precursor cells. Using this system we expected to express hFRS3 in neural tissues (the developing neural tube, brain and neural crest) between approximately E9 and E14. Of 9 hFRS3 lines established, 3 were maintained for study, 2 of these lines are still maintained (designated 22P3 and 1I3), while 1 line (designated 23B2) stopped breeding in the sixth generation (P5). Lines were maintained as heterozygous animals, as all homozygotic animals appeared lethal and were never carried to term. All data presented are representative of data from heterozygous animals from all three lines unless stated otherwise. Neural crest experimental data are derived from the 22P3 and 113 lines only.

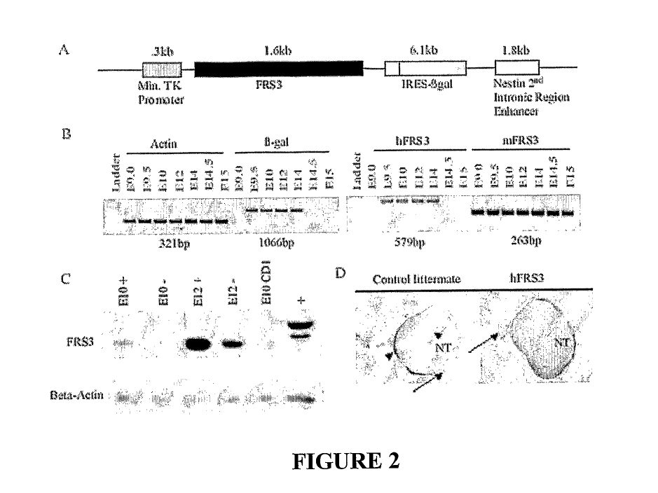

To determine the timing of construct expression in embryonic development we examined hFRS3, β-gal and control (murine FRS3, actin) gene expression in hFRS3 transgenic E9 to E15 embryos by RT-PCR (FIG. 2B). Transcripts for hFRS3 and β-gal were detected as early as E9.5 and persisted until E14. Earlier than E9.5 and later than E14, transgenic hFRS3 and β-gal transcripts were not detected by RT-PCR, while control transcripts were. Similarly, in situ hybridization for β-gal transcripts indicated expression from E9.5 through E14, but not at E15. When embryos were stained with X-gal, there was no β-gal activity detected, and on subsequent sequencing of genomic DNA from the lines we determined that the β-gal gene had a one base pair insertion at the 5′ end, resulting in transcript production without translation of fall-length active protein.

For FIG. 2: A: Schematic of the microinjection construct to make FRS3 transgenic mice. B: Vector transcript expression in E9.0 through E15 FRS3 transgenic animals by RT-PCR. Controls include actin and murine FRS3. Human FRS3 and β-galactosidase are transcripts derived from microinjected constructs showing expression of the transgene between E9-E14. C: Western blots showing FRS3 expression in brain and spinal cord lysates (5 μg total protein) from E10 and E12 hFRS3 positive and negative littermates. Negative control is CD1 E10 brain and spinal cord lysate, while positive control is 20 μg whole cell lysate from HEK293T cells over-expressing FRS3-myc-his. Actin re-probe of the same membrane is shown in the lower panel. D: Immunohistochemistry for FRS3 in control and FRS3 animals at E12. Note increased expression throughout all layers of the neural tube (NT) and dorsal root ganglion (arrow) compared to control (expression around the outside of the marginal layer) and the inner ventricular surface (arrowheads).

In situ hybridization results agree with those of [23], who expressed β-gal under the same Nestin second intronic enhancer. Expression was limited to the ventricular zone of the NT of the developing brain and spinal cord between E9.5 and E14.5, with expression from E10 through E14.5 in the DRG as well. No expression of the β-gal transcripts was seen outside the central nervous system and the DRG.

Histology: Adult animals and embryonic day 14 (E14) embryos were examined histologically for any gross abnormalities. Adult FRS3 transgenic animals displayed gross morphological abnormalities in the lung, liver and uterus. Liver phenotype indicated hydropic degeneration, commonly seen with hepatitis. Mice were tested for both parasitic and viral hepatitis and confirmed negative (data not shown). Lung tissue had small alveoli, but animals were not in apparent distress and were normally active, suggesting that this phenotype was asymptomatic. The uterus was grossly enlarged in diameter, while being truncated in length, resulting in a worm-like appearance of the entire organ, and the lumen was divided into a series of chambers by bands of muscle extending across from a thin myometrial layer (data not shown). Litter sizes were small (9 animals, p<0.05 compared with CD1 controls). Ovulation was apparently normal, with 12-15 oocytes released from both ovaries at mating, while litter sizes were significantly smaller. At E7, implantation sites were examined with an average of 9 implantation sites per pregnancy. The number of resorbing embryos was not significantly different from CD1s (FRS3: 2 embryos in 85 pregnancies; CD1: 3 embryos in 85 pregnancies). These data clearly show there was a failure of a portion of embryos to implant, probably due at least in part to the change of uterine morphology.

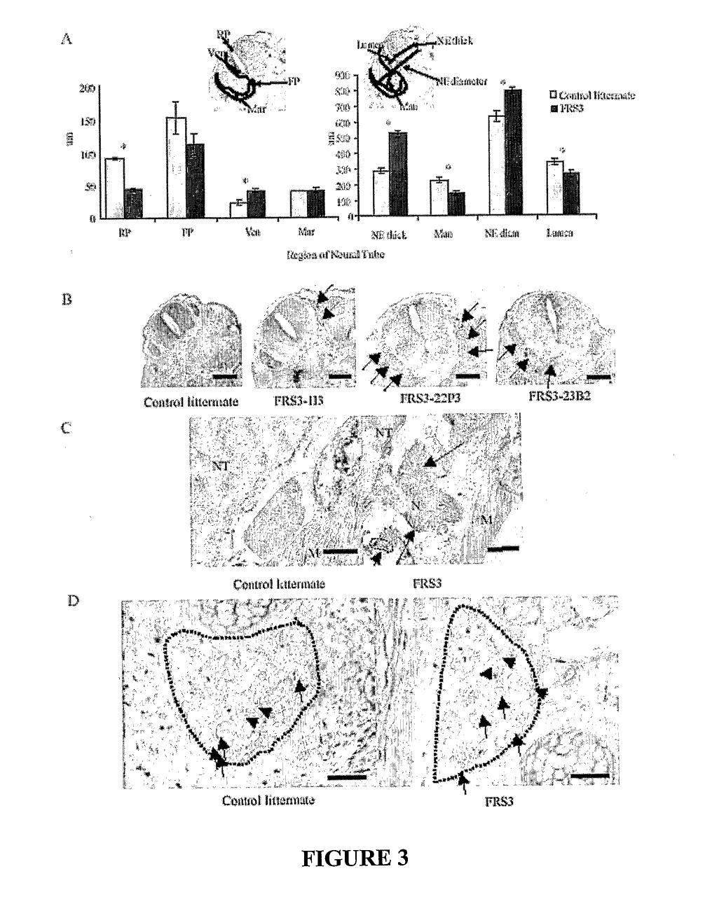

Embryonic defects were found in the head, NT, DRG and several tissues. Head phenotype was found only in homozygotic animals and will be described elsewhere (to be published elsewhere). Data presented here are from heterozygous animals and are representative of all three transgenic lines. In the spinal NT at E14, hFRS3 transgenic animals displayed statistical decreases in the thickness of the neural tube roof plate (p=0.0001), mantle layer (p=0.0022), overall thickness of the neuroepithelium (p=0.0059), and a decrease in the width of the neural tube lumen (p=0.0364; FIG. 3A). The ventricular layer of the neural tube was statistically wider in FRS3 over-expressing animals than in negative littermate controls (P=0.0012; FIG. 3B). While spinal NT phenotype appeared minimal there were marked changes found in the DRG, and consequently we focussed on the DRG.

For FIG. 3: A: Physical measurements within the neural tube in E14 negative littermates and FRS3 animals (n=15) at T4 level of the developing spinal cord (RP: roof plate; FP: floor plate; NE thick: overall thickness of neuroepithelium; Ven: ventricular layer; Man: mantle layer; Mar: marginal layer; NE diameter: overall diameter of the neural tube; Lumen: diameter of luminal opening within neural tube; *=statistically different within groups). B: DRG duplications and triplications found in FRS3 transgenics (1I3, 22P3, 23B2) at E12 (arrows). Scale bar=100 μm. C: Longitudinal sections of DRG's from CD1 and a FRS3 transgenic (P0). Arrows show duplicated ganglia adjacent to nerve tract. Arrowhead is a degenerating ectopic DRG lacking nerve tract support. NT=neural tube; M=muscle. Scale bar=200 μim. D: Toluidine blue stained E16 dorsal root ganglia showing large neuronal cells (arrows) and smaller diameter cells (arrowheads). Scale bar=50 μm.

The DRG exhibited duplications and triplications of entire DRG structures (FIGS. 3B, 3C). Individual DRG structures appeared grossly normal. Ectopic DRG were situated ventrally and medially to the ventral portion of normally positioned DRG (FIG. 3B, C). These duplicated DRG were similar in morphology to normal DRG in the dorsal-ventral plane and rostral-caudal axis between E10 and E14. Duplicated DRG were present at most but never at all levels of the neural tube, most often duplications were seen in the trunk region between T2-T4 level, and triplicated DRG were most commonly seen in the low lumbar and high sacral regions (L10-S5). Ectopic DRG were rarely seen caudal to S5.

At P0 (day of birth), ectopic DRG persisted in most FRS3 over-expressing mice. Those duplicated DRG that were located adjacent to main nerve tracts of normally placed DRG structures appeared to be persistent (FIG. 3C), while those that were not adjacent to nerve tracts were very small and apparently decreasing in both size and cell number. At P0, normally situated DRG in duplicated structures with well-developed nerve tracts commonly exhibited altered morphology, appearing to be lobed or oddly-shaped compared to the more oval shape of controls. Nerve tracts commonly coursed between these ganglia, contacting both ganglia, as opposed to contacting only one end of one ganglion (FIG. 3C).

When examined at the cellular level it was found that the majority of cells populating E14 DRG of hFRS3 over-expressing mice were of small diameter neuronal phenotype (FIG. 3D). There was a small proportion of large diameter cells of neuronal phenotype present (10-12% of total cells, compared to controls with 15-16% of total cells) while the remainder of the DRG was composed of small diameter cells.

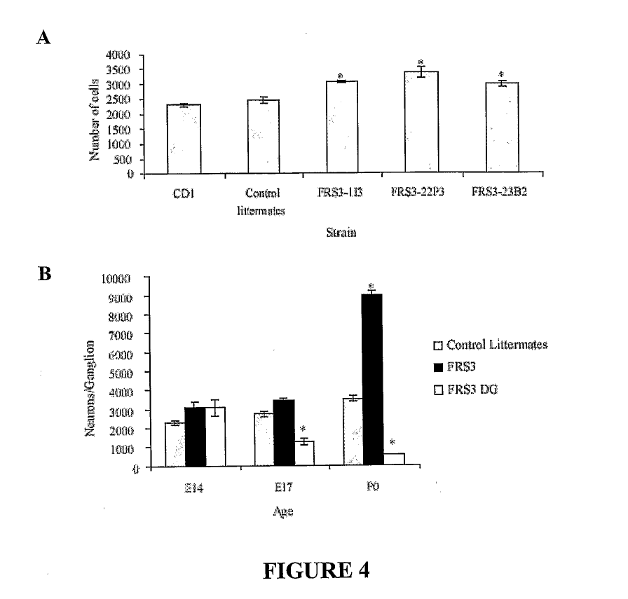

Cell counts: We chose to concentrate cell counts at the T4 level, as this level most often showed duplications in DRG, and to maintain consistency as numbers of neurons within DRG change depending on spinal column level. Total cell counts were performed on single DRG structures and are presented as number of cells per single DRG structure (FIG. 4A). All cells with visible nuclei were counted across serial sections through entire DRG structures and counts were adjusted as per the method of Abercrombie [25] to reduce sampling error. Negative littermate controls were animals from the same litters as experimental animals, tested by RT-PCR and negative for human FRS3 transcripts. Control animals (CD1) and negative littermates had 2287±70 and 2464±116 cells per ganglion (not statistically different and consistent with data by [26]), while each of the FRS3 over-expressing lines had significantly higher numbers of cells (3086±59, p<0.001; 3368±177, p<0.001 and 2986±93, p<0.01; see FIG. 4A). The 35-50% increase in cell number appeared to be due to an increased number of small diameter cells. Large diameter cells present in CD1 and control littermate animals were not statistically different (366±11 (16%) and 370±17 (15%), respectively). The total number of large diameter cells in FRS3 over-expressing animals was consistent with controls (375±9, 356±26, and 361±15) resulting in a lower overall percentage of large diameter cells per DRG (10.5%, 12% and 12% per line) due to the increase in gross cell number per ganglion. At P0, DRG of control animals contained 3514±461 cells, while in hFRS3 animals those DRG structures that were adjacent to nerve tracts had a significant increase (p<0.001) in cell number to 9019±168 cells. Those duplicated DRG structures at distance from nerve tracts in hFRS3 animals contained only 597±3.5 cells, significantly less (p<0.001) than control DRG (FIG. 4B). These data show that hFRS3 transgenic animals exhibit an increase in cell number in DRG, as well as an increase in small diameter morphology cells, suggesting a role for FRS3 in DRG development.

For FIG. 4: A: DRG cell counts across three FRS3 over-expressing lines established from independent microinjections compared to CD1 and negative littermates at E14 (*=statistically significant between groups). B: DRG cell counts averaged across the three strains between E14 and P0 (*=statistically significant within groups; FRS3 DG=ectopic duplicated ganglia seen in FRS3 transgenic animals not associated with a nerve tract).

Extension of the developmental program: The DRG are formed by neural crest (NC) cells that delaminate from the dorsal neural tube and migrate to a ventrolateral position beside the NT by approximately E10 in the mouse. There are several ways that DRG abnormalities in hFRS3 transgenic mice could have arisen. First, changes in migration could have resulted in non-DRG-fated NC being recruited into populating the DRG resulting in increased cell number. Alternatively, NC fated to reside in the DRG may be proliferating while migrating or after populating the DRG, resulting in the increase seen in cell number without recruitment of other NC populations. Another possibility is that there is an increased number of NC delaminating from the NT and coming to reside in the DRG. To address these possibilities, we have examined markers for NC during migration, assessed proliferation occurring in the DRG throughout embryonic development of these mice and delamination of NC from the NT.

To determine what effect FRS3 over-expression might have on the NC, several markers known to be expressed in NC during development were investigated for mRNA expression by ini situ hybridization. Negative littermates and CD1 animals were examined in parallel with FRS3 over-expressing animals to provide controls. Early markers Snail, Pax3, bone morphogenic protein (BMP) 2 and BMP4 are all known to be involved in the selection of the neuroepithelium that will delaminate as the NC and migrate off the tube ([27]; [28]; [29]; [30]; [31]). From E9.5 through E12, Pax3, Snail, bone morphogenic protein (BMP) 2 and BMP4 mRNA expression were examined and found to be normal (data not shown). As Pax3 and Snail were unaffected, and phenotype manifested only in the DRG, we did not pursue the expression of any known signaling proteins involved in melanocyte formation as the populations forming melanocytes and DRG are reported to be different in development ([32]; [33]).

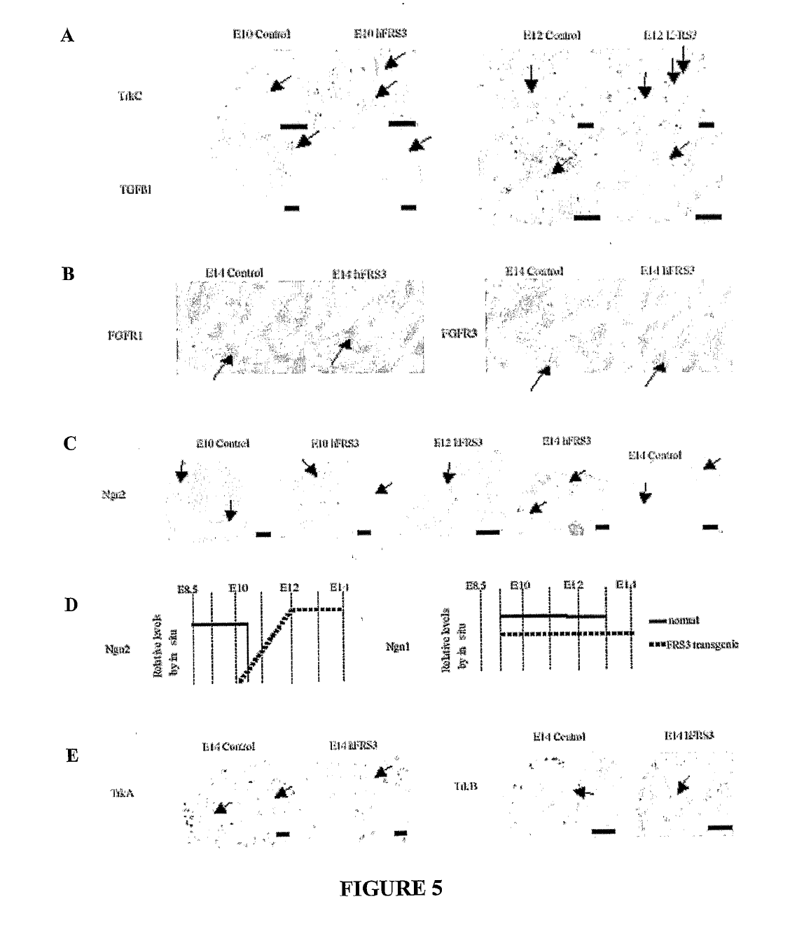

As duplicated DRG structures may have arisen due to an over-population of NC through increased or maintained proliferation, TrkC, FGFR1 and TGFβ1 were examined (FIG. 5). These proteins are known to directly affect proliferation of NC cells, with TrkC and FGFR1 eliciting a proliferative effect while TGFβ1 is known to decrease proliferation ([34]; Zhang et al., 1997; [35]; [36]). TrkC expression was found to be much higher at E10 and remained high through E14. FGFR1 and TGFβ1 were both decreased from control levels at both stages. As FRS3 is over-expressed, the decreased expression of FGFR1 could be due to receptor down-regulation due to increased signaling via the FRS3 adapter protein. The increase in TrkC expression with decreased TGFβ1 strongly suggests that the increase in number of cells populating the DRG may arise through increased proliferation, which is discussed further below.

For FIG. 5: A: In situ hybridization for TrkC and TGFβ1 in E10 and E12 dorsal root ganglia showing changes in transcript expression in FRS3 transgenic animals. B: Immunohistochemistry for FGFR1 and FGFR3 (E14) in control and FRS3 transgenics. C: Neurogenin 2 expression from E10 through E14 in control and FRS3 transgenic DRG. D: Schematic representation of normal (solid lines) and hFRS3 transgenic (dashed lines) expression levels of neurogenin 2 (Ngn2) and 1 (Ngn1). E: TrkA and TrkB expression changes in dorsal root ganglia of E14 embryos. Photos are inversed so signal is black, Scale bar=100 μm. Arrows point at dorsal root ganglia.

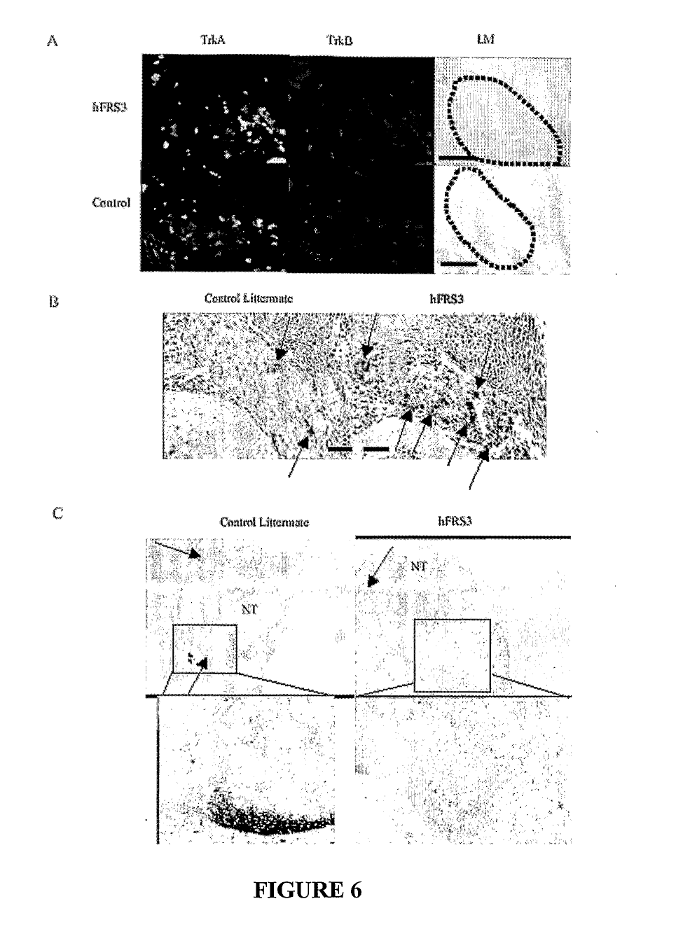

With the apparent change in cell number and prevalence of small diameter cells in the DRG, we also chose to examine markers known to be expressed in fate restricted DRG sensory neuron development, including neurogenin 1 (Ngn1) and neurogenin 2 (Ngn2) (FIG. 5), as well as the markers expressed by differentiated DRG neurons, TrkA and TrkB (FIG. 6). Neurogenin 1 expression is required for formation of small diameter TrkA positive cells of the DRG, while Ngn2 expression fate restricts to the TrkB positive lineage [37]. If Ngn1 and Ngn2 are knocked out, failure of DRG development occurs [37]. Each of these genes showed abnormalities in expression levels of mRNA. Neurogenin 2 expression should be finished by E11 [37]; however, levels in the FRS3 over-expressing mice were elevated at E12, persisted to E14, and then decreased completely by E14.5 (data not shown). Neurogenin 1 expression was decreased compared to controls, but persisted at low levels to E14.5 (normally Ngn1 expression ceases at approximately E13, with expression dependent on earlier Ngn2 expression; [37]). TrkA expression in the DRG was decreased significantly, while TrkB expression was very high, detected in almost all cells. When examined closer, the small diameter cells accounting for the 35-50% increase in DRG cell population appeared to be positive for TrkB and not TrkA. Larger diameter cells were TrkB positive, as expected, as medium and large diameter neurons are reported to be TrkB expressing and the first population of cells to differentiate in the DRG [38]. By E14-15 there should be a large proportion (approximately 40-45%) of small diameter cells that are TrkA positive [39]. There were very few TrkA only positive cells in FRS3 over-expressing mice (10-20 cells per ganglion examined at E14) suggesting that either these cells are wrongly fated, or that the developmental program leading up to TrkA expression has been delayed in hFRS3 transgenic mice. When examined at P0, DRG neurons were found to be TrkA positive, or TrkB positive, with many smaller diameter cells being TrkB positive (FIG. 6A). Normally, small diameter cells are mainly TrkA positive, but in hFRS3 transgenic animals it appears that hFRS3 has an effect on the fating of these neurons to TrkB or that some other system has been affected by FRS3 and is itself producing a change in fate.

For FIG. 6: A: TrkA and TrkB in situ hybridization in P0 FRS3 animals and control littermates, showing overlap of TrkB expression with TrkA in many cells in FRS3 animals (LM=light micrograph; dotted line denotes the outline of the dorsal root ganglion). B: Ki67 immunohistochemistry to detect proliferating cells in the E14 dorsal root ganglion (arrows; positive signal is black). Scale bars=50μm. C: Expression of the Wnt receptor, Frizzled2, in E12 control and FRS3 animals. Note the increased zone of positive cells in the developing spinal cord in FRS3 animals, and increased expression in FRS3 dorsal root ganglion compared to control (arrows).

With changes in FGFR1, TGFβ1 and Trk C expression, transcription factors Ngn1 and Ngn2, Trk receptor expression, as well as the persistent population of small diameter cells in DRG, we investigated the continuation of proliferation in DRG to determine if this event had also been extended into times when it should be completed. The antigen Ki67 is expressed only in actively proliferating cells and can be used to determine the proliferative status of cells ([40]; [41]). When sections through the DRG and NT of control E14 animals were examined, Ki67 was present in the ventricular region of the NT in control animals and at E10-12 in DRG (FIG. 6B). Embryonic stage 14 hFRS3 over-expressing animals showed many Ki67 immunoreactive cells in the DRG, as well as in the immediate surrounding mesenchyme of the NT (FIG. 6B) evidence that proliferation is still occurring in DRG of these animals at a level higher than controls. This supports in situ data suggesting maintenance of proliferation by FRS3, possibly via TGFβ1 and TrkC expression changes. Taken together, it appears that the increase in cell number in hFRS3 transgenic DRG is due to increased and prolonged proliferation of DRG-fated NC.

The Frizzled proteins are receptors for the Wnts, proteins involved in the patterning of the central nervous system. Frizzled is known to be expressed in the developing central nervous system, in the ventral neural tube in the mouse by E11.5. The cells that are positive for Frizzled were found to be cells that are limited to neural restricted precursor cells [42]. We investigated the expression of Frizzled in the central nervous system of hFRS3 over-expressing mice and negative littermates to determine if over-expression of hFRS3 could induce an expansion of neural restricted precursor cells (as evidenced by an increase in Frizzled immunoreactivity). We demonstrated that an increase in Frizzled positive cells in the ventral neural tube due to the over-expression of hFRS3 (FIG. 6C). We found a drastic increase in the immunoreactivity of the entire axis of the developing spinal cord and developing brain (brain data not shown) up to and including E14. Representative sections are shown in the attached figure-positive cells are brown in colour from the DAB substrate reaction. Surprisingly, expression was not restricted to the ventral region of the neural tube as expected, extended throughout most of the dorso-ventral axis of the neural tube, seemingly excluding only the region of the roof plate and extreme dorsal neural tube. Increased Frizzled positive cells in hFRS3 transgenic animals over controls shows an expansion of the neural restricted precursor cell population within the neural tube. We show that these precursors are displaced or have migrated throughout the tube during development and likely include the entire population of cells responsible for neurogenesis throughout all layers of the developing cord and brain.

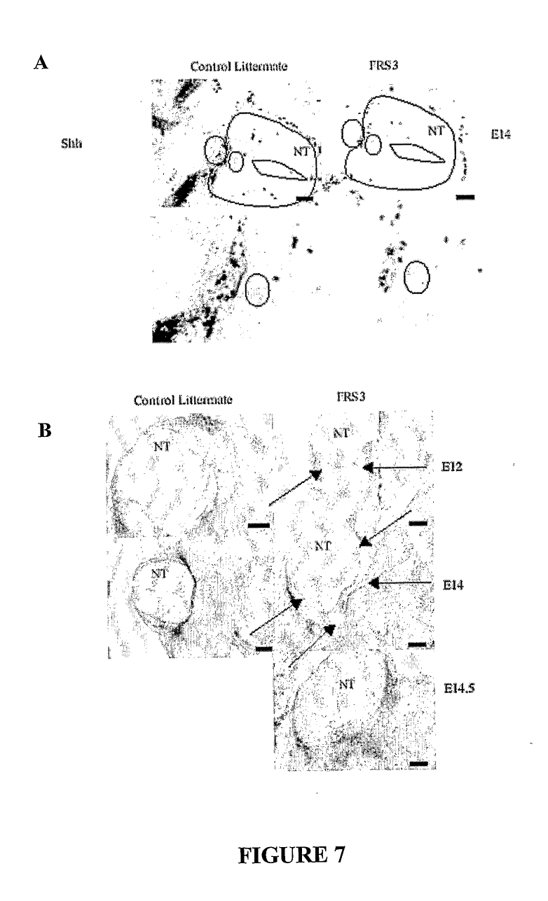

Neural crest migration in hFRS3 mice: In studies performed in Shh knockout mice, DRG phenotypes were seen that closely paralleled the phenotype seen in hFRS3 mice. With published reports of the complex interplay between Shh and FGF signaling, we chose to examine the expression of Shh. Sonic hedgehog is normally expressed in the ventral neural tube and notochord (for Shh review see [43]). In hFRS3 mice, Shh expression in the ventral neural tube was decreased over control animals at E10 (FIG. 7A), but returned to normal levels by E14.5. A secreted form of Shh is known to elicit the production of versican family proteoglycans in surrounding tissues which are at least partly responsible for directing NC migration in the head by limiting cell mobility [44]. Due to the decrease in Shh expression, and the fact that the versican expression appears to be controlled by long-range Shh signaling, immunohistochemistry for versican was performed. We found that there was a marked reduction in the amount of versican surrounding the ventral portion of the NT in hFRS3 mice from E10 through E14. At E14.5, versican staining appeared to return to normal levels, comparable with control littermate and CD1 embryos (FIG. 7B). Immunoreactivity for versican appeared to be reduced but not absent between the medially portion of the ventral NT and the developing vertebrae, consistent with our observations that ectopic DRG structures were never found directly in the midline of the embryo, unlike reports of ectopic trigeminal ganglia formation in Shh knockout mice [44].

For FIG. 7: A: Fluorescent in situ hybridization at E10 for sonic hedgehog (Shh) showing ventral neural tube and notochord expression in control littermates and decreased expression in FRS3 animals (circles show areas of Shh expression). Lower panels are increased magnification of upper panels. B: Versican immunohistochemistry in control littermates and FRS3 transgenic animals. Arrows point to locations where versican is reduced in E12-E14 FRS3 animals.

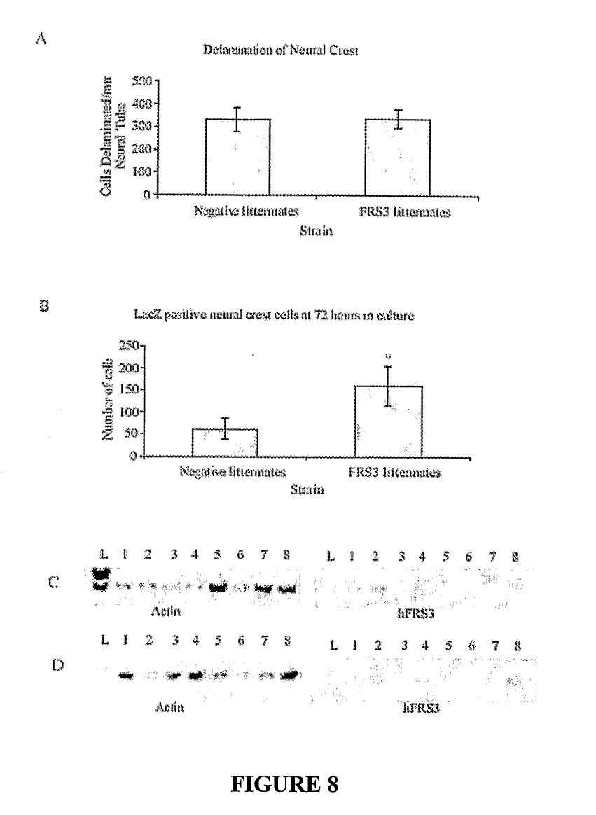

Neural crest cultures: Neural crest cultures were prepared from E9 negative and hFRS3 expressing littermates, and cultured for 72 hours. We examined the possibility that there were more NC delaminating from the NT in FRS3 animals, which could account for the increased number of cells apparently residing in the DRG. The total number of cells delaminated and the length of the NT were correlated to give the number of cells delaminating per millimeter of tube. The results showed that there was no statistical difference in the number of NC delaminating from the NT of hFRS3 over-expressing mice (333±52) or their control littermates (341±39; see FIG. 8A) suggesting that there is no change in the process of delamination in hFRS3 transgenic animals and that extra NC delamination from the NT was not causing the increase in cells ultimately residing in the DRG.

For FIG. 8: A: NC cell delamination in FRS3 and control littermate E9 embryos in culture. Values are not statistically different. B: NC cell proliferation in FRS3/LacZ and control E9 embryos at 72 hours in culture. Values are statistically different (p<0.00005). C: PCR of genomic DNA from FRS3 and control littermate E9 embryos used in above neural crest cultures for actin (positive control and FRS3). D: RT-PCR of RNA from embryos used in NC cultures showing actin (left) and FRS3 (right) expression after 72 hours of culture. Samples are the same as genomic DNA in panel C.

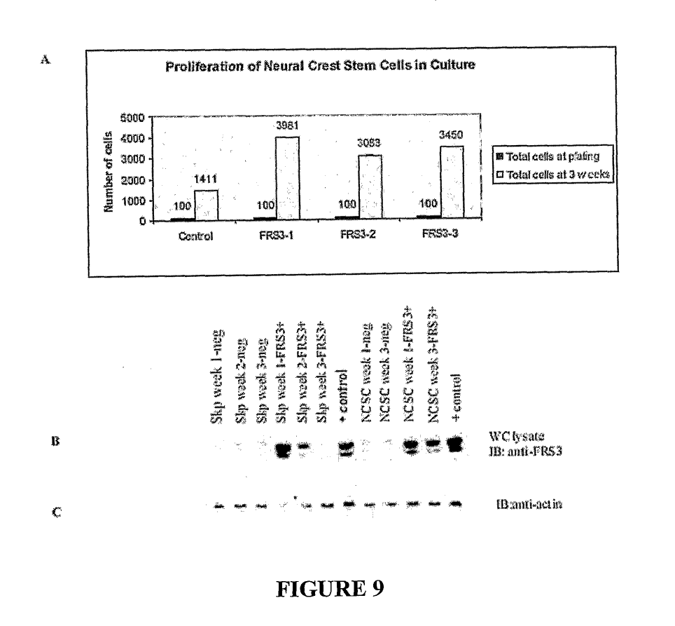

Cells were counted again at 72 hours, and then stained with X-gal. There was a slight increase in cell number per NT length in negative littermate controls from 333±52 cells to 381±50 while hFRS3 NC preps contained 560±45 cells when correlated with tube length. At 72 hours, there were on average 62±24 (19%) X-gal positive cells in negative littermate dishes compared with 160±44 (29%) in FRS3 littermate cultures (FIG. 8B). Cells at 24 hours have not been X-gal stained, but as delamination is occurring prior to the onset of expression of the construct, and expression of Snail, Pax3, BMP2 and BMP4 were normal, we expect there to be no difference between control and FRS3 transgenic embryos. The increase in X-gal positive cells at 72 hours represents a 2.6-fold increase in cells expressing our construct in hFRS3 transgenic animals over 3 days in culture. When the cells are kept in culture for up to 3 weeks, the FRS3 expressing NSCS's continue to generate an approx. 2-3 fold more number of cells in vitro (FIG. 9A). An adult DRG from hFRS3 transgenic animals show a 2.6-fold increase in cell number in ganglia that persist on nerve tracts. Taken together these data strongly suggest that the increase in number of cells in DRG is due to the increased proliferation of a subset of NC cells that continue to proliferate after delamination has occurred. It also suggests that the crest are at least partially predetermined to become DRG within 72 hours of culture as not all NC are X-gal positive at this time. This also shows that expression of FRS3 under the second intronic enhancer of the Nestin gene is capable of targeting a subset of NC cells destined for the DRG and not all NC cell populations.

For FIG. 9: FRS3-Induced FGF-Dependent In Vitro Proliferation of Neural Crest-Derived Stem Cells after 3 weeks in Culture. (A) Total cell counts of NCSC's from control as compared to FRS3 expressing mice. (B) Western blot showing increased expression of transgenic FRS3 at 1 week and 2 weeks in vivo in NCSC's or skin-derived precursor cells (SKPs). By 3 weeks in culture, expression of FRS3 in the SKPs is decreased to levels comparable to controls but high levels are still detected in NCSC's. (C) Control blot showing comparable levels of protein loaded in each lane.

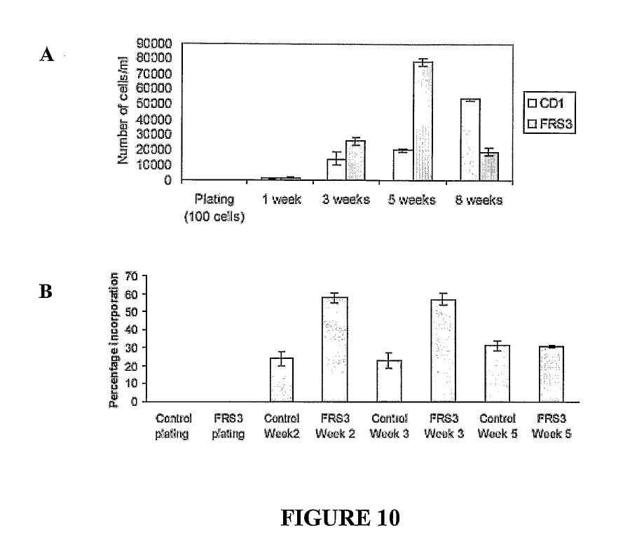

With the recent identification that Nestin is also expressed in skin-derived precursors (SKPs, [6]), we determined whether our Nestin-hFRS3 expressing transgenics would also show increased proliferation of SKP's in culture relative to controls. As shown in FIG. 10A, FRS3 does increase the number of SKP's generated in culture, up to 5 weeks. Under the current conditions, no increase in proliferation is observed after 8 weeks (FIG. 10A), consistent with a decreased rate of incorporation of the thymidine analague, BrdU, after 3 weeks (FIG. 10B) and the decreased expression of hFRS in the cultured SKPs after 3 weeks in culture (FIG. 9B).

For FIG. 10: Effect of FRS3 on the FGF-Dependent In Vitro Proliferation of Skps. (A) Skp's derived from Nestin-FRS3 expressing transgenic mice increase numbers of Skp's by approximately 4-fold by 5 weeks in culture. The expression of FRS3 is subsequently lost in these cells and so are the numbers of cells. (B) Skp's derived from FRS3 transgenics show increased BrdU encorporation (a marker of cellular DNA replication) at 2 and 3 weeks in culture.

To determine whether precursor cells could be infected with a vector or virus designed to over-express FRS3, we assayed SKP's isolated from normal CD1 mice for changes in FGF-dependent proliferation following infection with an adenovirus designed to over-express hFRS3 (Li and Meakin, unpublished data). As demonstrated in Table 1, we observed and approx. 50-80% increase in the numbers of SKPs, at 72 hours in culture, following infection with Ad-hFRS3 relative to a control Ad virus expressing β-galactosidase.

Table 1 indicates the proliferation in SKPs infected with β-galactosidase or hFRS3 expressing adenovirus (cell counts of infected cells after 48 hours infection and again at 72 hours).

TABLE 1

| | | # cells at 0 | # cells at 72 | |
|---|---|---|---|---|
| | | hours post | hours post | % |
| Adenovirus | MOI | infection | infection | Increase |
| β-galactosidase | 25 | 2500 | 2600 | 4% |
| (Control) | 50 | 3000 | 3000 | 0% |
| | 75 | 3200 | 3200 | 0% |
| | 100 | 3200 | 0 (toxic) | NA |
| | 150 | 3000 | 0 (toxic) | NA |
| hFRS3 | 25 | 3500 | 6100 | 74% |
| | 50 | 3700 | 5500 | 48% |
| | 75 | 4500 | 8300 | 84% |
| | 100 | 3200 | 0 (toxic) | NA |
| | 150 | 2500 | 0 (toxic) | NA |

We have generated mice that over-express hFRS3 under the Nestin second intronic region enhancer and minimal TK promoter. When using this system phenotypic abnormalities were introduced into these mice. We show over-population of DRG structures in FRS3 over -expressing animals, formation of ectopic ganglia, with maintenance of ectopic ganglia in contact with nerve tracts later in development demonstrating a central role for signaling via FRS3 in these processes.

There are several possible ways that over-population of the DRG and ectopic ganglia could occur: First, there may be increased delamination and thus migration of greater numbers of NC to ectopic locations after proliferation has occurred. Second, NC could arrive at ectopic locations and proliferation continues to produce a ganglion of abnormal size. Third, a combination of these two processes may be responsible. Our current study shows proliferation in NC during migration, as well as in formed DRG as late as E14, suggesting that proliferation is prolonged and occurring within DRG after they are established. Other trunk structures formed from NC such as the sympathetic and enteric ganglia appeared normal in hFRS3 animals, strongly suggesting that recruitment from these NC was not occurring to add to the population destined for DRG, but that extra population was derived from proliferation or increased delamination from the NT. We have ruled out increased NC delamination from the NT, and have shown evidence of increased proliferation of NC cells. These data clearly demonstrate an increase in proliferation of DRG-fated NC. In older animals, DRG that are not supported by nerve tracts degenerate, strongly suggesting that these ganglia form in completely ectopic locations, and are unable to establish trophic support from targets. Those structures able to establish nerve tracts appear to have high cell survival and persist with increased cell numbers. Fibroblast growth factor signaling is known to promote proliferation of NC ([34]; [45]; [35]; [36]) which agrees with our findings in hFRS3 transgenic mice having more cells populating the DRG. For the population of NC which will form the DRG, proliferation is thought to be β-FGF driven, requiring FGFR1 to be expressed on migrating cells and β-FGF presence in the environment with expression of a neurotrophin ([34]; [45]). Up-regulation of TGFβ1 results in negative regulation of FGF-mediated proliferation [45]. Concomitant with this is expression of receptors allowing sensitivity to select neurotrophins, which results in differentiation into either TrkA, B or C positive cells depending on their fate restriction ([34]; [36]). FRS3 is known to interact with FGF-activated FGFR1. By over-expressing FRS3 we show it is possible to increase FGF signaling in these cells without increasing FGFR expression, producing maintenance of the proliferating state for a period of time that corresponds with construct expression. Over-expression of FRS3 in the NC population while migrating and shortly after DRG condensation would account for the increase in cell number seen in DRG from FRS3 over-expressing animals. The decrease in TGF-β in hFRS3 animals shows there is also down-regulation of inhibition causing maintenance of proliferation, delineating a role for FRS3 in the maintenance of proliferation.

We have also shown that in hFRS3 over-expressing mice there is a clear decrease in Shh expression with concomitant changes in Ngn expression suggesting that all these pathways are linked, supporting data by Ota and Ito [46]. Perez et al [47] have shown that migrating neural crest expressing Ngn2 appear committed to sensory neuron fate when exposed to BMP2. Neural crest cells in hFRS3 transgenic mice express Ngn2 much longer than normal, have normal levels of BMP2 and would therefore contribute to sensory DRG neurons. Coupled with decreased versican expression, ectopic DRG structures are found each exhibiting increased cell numbers over controls.

It has been shown in trigeminal ganglia that the expression of FGF2, and signaling via FGFR1 (which is known to interact with FRS3), can cause the decrease of Ngn expression [46]. Sonic hedgehog expression has no effect on Ngn2 expression in the same system [46] but appears to be required for Ngn1 expression. These observations are consistent with hFRS3 over-expression causing decreased Ngn1 expression, but not decreasing Ngn2 expression if in fact Shh is involved in the Ngn1 expression cascade. As hFRS3 transgenic mice show a decrease in Shh, we would expect to see decreased Ngn1 expression that would return to normal upon Shh returning to normal at E14.5. Neurogenin 2 expression is prolonged until E14.5 in transgenic animals, where in normal animals Ngn2 expression decreases by E11 [37]. There is an interplay between Ngn2 and Ngn1 expression, with Ngn2 expressed earlier (E9-E10.5) in the mouse embryo, and Ngn1 expression requiring Ngn2 expression [37]. In addition, Ngn1 expression has been shown to also require Shh expression in the trigeminal ganglia, while Ngn2 expression occurs independent of Shh [46]. Human FRS3 transgenic mice exhibit an abnormally long period of Ngn2 expression, with decreased Ngn1 expression, both which appear to return to normal upon transgene inactivation. Sonic hedgehog expression is clearly down-regulated in these transgenic mice, which would agree with the delay in Ngn1 expression while Ngn2 expression is present.

Expression of the construct persists until E14, after which time hFRS3 expression is no longer detected. This decrease in FRS3 is concurrent with an increase in Shh expression. This evidence also strongly suggests that hFRS3 expression has an effect on the expression of Shh, as Shh is up-regulated to normal levels upon cessation of construct expression. Multiple proteoglycans have been implicated in the control of NC migration, including aggrecan and collagen IX ([48]; [49]; [50]) and direct interactions between the hedgehog family and proteoglycans have been shown in tooth development [51]; [52].

Human FRS3 over-expressing mice did not display single DRG structures located in the midline, as would be expected if all versican expression was affected. There are two main locations of Shh expression in the embryo, the ventral neural tube, and the notochord (for review see [43]). Versican immunohistochemistry does show a low expression of versican located in the midline of hFRS3 transgenic animals, ventral to the neural tube in the region that correlates with the notochord. Expression of hFRS3 in the neural tube appears to have decreased Shh expression only in the neural tube, while the notochord expression is still normal and able to elicit versicans in this area. This would prevent migration of NC into the midline, but still permits ectopic laterally located ventral DRG. This is consistent with expression of the construct. We have used the Nestin second intronic region enhancer so expression is limited to NT [23]. Taken together, the decrease in Shh signaling appears to be involved in the final location of ectopic DRG seen in these embryos.

We have shown that by over-expressing FRS3, an adapter protein involved in signaling from the FGF receptor family, that we can influence the number of cells populating the DRG. We have also shown an effect on versican expression, which correlates with the formation of ectopic DRG seen in these mice. Ectopic DRG persist past birth provided contact with nerve trunks was established. Over-expression of hFRS3 also leads to increased proliferation, and an extension in developmental program in DRG-fated NC cells, the latter of which manifests in alterations of cell fate. Taken together, these data demonstrate that FRS3 functions in the maintenance of proliferation early in sensory neuron precursor cells, and additionally, later in development can influence the fating of these cells during differentiation.

As can be understood by one skilled in the art, many modifications to the exemplary embodiments described herein are possible. The invention, rather, is intended to encompass all such modification within its scope, as defined by the claims.

### REFERENCES

1. Pevny, L. and M. S. Rao, The stem-cell menagerie. Trends Neurosci., 2003. 26: p. 351-359.
2. Kondo, M., et al., BIOLOGY OF HEMATOPOIETIC STEM CELLS AND PROGENITORS: Implications for Clinical Application. Annual Review of Immunology, 2003. 21(1): p. 759-806.
3. Ahmad, I., et al., Neural stein cells in the mammalian eye: types and regulation. Semin. Cell Dev. Biol., 2004. 15: p. 53-62.
4. Chargé, S. B. P. and M. A. Rudnicki, Cellular and Molecular Regulation of Muscle Regeneration. Physiol. Rev., 2004. 84: p. 209-238.
5. Beltrami, A. P., et al., Adult Cardiac Stem Cells are Multipotent and Support Myocardial Regeneration. Cell, 2003. 114: p. 763-776.
6. Toma, J. G., et al., Isolation of multipotent adult stem cells from the dermis of mammalian skin. Nat. Cell. Biol., 2001. 3: p. 778-784.
7. Hama, J., et al., SNT-1/FRS2a physically interacts iwth Laloo and mediates mesoderm induction by fibroblast growth factor. Mech. Dev., 2001. 109: p. 195-204.
8. Kusakabe, M., et al., Xenopus FRS2 in involved in early embryogenesis in cooperation with the Src family kinase Laloo. Embo Reports, 2001. 2: p. 727-735.
9. Dono, R., Firbroblast growth factors as regulators of central nervous system development and function. Am. J. Physiol. Regul. Integr. Comp. Physiol., 2003. 284: p. R867-R881.
10. Ortega, S., et al., Neuronal Defects and Delayed Wound Healing in Mice Lacking Fibroblast Growth Factor 2. Proc. Natl. Acad. Sci (USA), 1998. 95: p. 5672-5677.
11. Raballo, R., et al., Basic Fibroblast Growth Factor (Fgf2) is Necessary for Cell Proliferation and Neurogenesis in the Developing Cerebral Cortex. J. of Neurosci., 2000. 20: p. 5012-5023.
12. Vaccarino, F. M., et al., Changes in Cerebral Cortex Size are Governed by Fibroblast Growth Factor During Embryogenesis. Nature Neurosci., 1999. 2: p. 246-253.
13. Hadari, Y. R., et al., Binding of Shp2 Tyrosine Phosphatase to FRS-2 is Essential for Fibroblast Growth Factor-Induced PC12 Cell Differentiation. Mol. Cell. Biol., 1998. 18: p. 3966-3973.
14. Meakin, S. O., et al., The Signaling Adapter Protein FRS-2 Competes with Shc for binding to TrkA: A Model for Discriminating Proliferation and Differentiation. J. Biol. Chem., 1999. 274: p. 9861-9870.
15. Xu, H., K. W. Lee, and M. Goldfarb, Novel Recognition Motif on Fibroblast Growth Factor Receptor Mediates Direct Association and Activation of SNT Adapter Proteins. J. Biol. Chem., 1998. 273: p. 17987-17990.
16. Easton, J. B., et al., Brain-derived Neurotrophic Factor Induces Phosphorylation of Fibroblast Growth Factor Receptor Substrate 2. J. Biol. Chem., 1999. 274: p. 11321-11327.
17. Dhalluin, C., et al., Structural basis of SNT PTB domain interactions with distinct neurotrophic receptors. Mol. Cell., 2000. 6: p. 921-929.
18. Ong, S. H., et al., FRS2 Proteins Recruit Intracellular Signaling Pathways by Binding to Divers Targets of Fibroblast Growth Factor and Nerve Growth Factor Receptors. Mol. Cell. Biol., 2000. 20: p. 979-989.
19. Dixon, S. J., et al., Trk Receptor Binding and Neurotrophin/Fibroblast Growth Factor (FGF)-Dependent Activation of the FGF Receptor Substrate (FRS)-3. J. Neurochem., 2005.
20. Xu, L. and M. Goldfarb, Multiple Effector Domains within SNT1 Coordinate ERK Activation and Neuronal Differentiation of PC12 Cells. J. Biol. Chem., 2001. 276: p. 13049-13056.
21. McDougall, K., et al., Developmental Expression Patterns of the signaling adapters FRS-2 and FRS-3 during early embryogenesis. Mechanisms of Development, 2001. 103: p. 145-148.
22. Hadari, Y. R., et al., Critical Role for the Docking-Protein FRS2 α in FGF Receptor-mediated Signal Transduction Pathways. Proc. Natl. Acad. Sci. (USA), 2001. 98: p. 8578-8583.
23. Lothian, C. and U. Lendahl, An evolutionarily conserved region in the second intron of the human nestin gene directs expression to the CNS progenitor cells and to early neural crest cells. Eur. J. Neurosci., 1997. 9(452-462).
24. Yaworsky, P. J. and C. Kappen, Heterogeneity of Neural Progenitor Cells Revealed by Enhancers in the Nestin Gene. Dev. Biol., 1999. 205: p. 309-321.
25. Abercrombie, M., Estimation of Nuclear Population from Microtome Sections. Anat. Rec., 1946. 94: p. 239-247.
26. Berg, J. S. and P. B. Farel, Developmental Regulation of Sensory Neuron Number and Limb Innervation in the Mouse. Brain Res. Dev. Brain Res., 2000. 125: p. 21-30.
27. Serbedzija, G. N. and A. P. McMahon, Analysis of Neural Crest Cell Migration in Splotch Mice using a Neural Crest-Specific LacZ Reporter. Developmental Biology, 1997. 185: p. 139-147.
28. LeDourain, N. M. and C. Kalcheim, The Neural Crest. 2nd Edition ed. 1999, New York: University Press.
29. Nieto, M. A., The early steps of Neural Crest Development. Mech. Dev., 2001. 105: p. 27-35.
30. Knecht, A. K. and M. Bronner-Fraser, Induction of the Neural Crest: A multigene Process. Nature Rev., 2002. 3: p. 453-461.
31. Villanueva, S., et al., Posteriorization by FGF, Wnt and retinoic acid is required for neural crest induction. Dev. Biol., 2002. 241: p. 289-301.
32. Luo, R., et al., Molecular Identificatin of Distinct Neurogenic and Melanogenic Neural Crest Sublineages. Dev., 2003. 130: p. 321-330.
33. Aoki, Y., et al., Sox10 regulates the development of neural crest-derived melanocytes in Xenopus. Dev. Biol., 2003. 259: p. 19-33.
34. Murphy, M., et al., FGF2 regulates proliferation of neural crest cells with subsequent neuronal differentiation regulated by LIF or related factors. Dev., 1994. 120: p. 3519-3528.
35. Sieber-Blum, M. and L.-M. Zhang, Growth factor action in neural crest diversification. J. Anatom., 1997. 191: p. 493-499.
36. Sieber-Blum, M., Growth Factor Synergism and Antagonism in Early Neural Crest Development. Biochem. Cell Biol., 1998. 76: p. 1039-1050.
37. Ma, Q., et al., NEUROGENIN 1 and NEUROGENIN2 control two distinct waves of neurogenesis in developing dorsal root ganglia. Genes and Dev., 1999. 13: p. 1717-1728.
38. Fariñas, I., et al., Characterization of Neurotrophin and Trk Receptor Functions in Developing Sensory Ganglia: Direct NT-3 Activation of TrkB Neurons In Vivo. Neuron, 1998. 21: p. 325-334.
39. Mu, X., et al., Neurotrophin Receptor Genes are Expressed in Distinct Patterns in Developing Dorsal Root Ganglia. J. Neurosci., 1993. 13: p. 4029-4041.
40. Gerdes, J., et al., Production of a Mouse Monoclonal Antibody Reactive with a Human Nuclear Antigen Associated with Cell Proliferation. Int. J. Cancer, 1983. 31: p. 13-20.
41. Gerdes, J., et al., Cell Cycle Analysis of a Cell-Proliferation-Associated Human Nuclear Antifen Defined by a Monoclonal Antibody Ki67. J. Immunol., 1984. 133: p. 1710-1715.
42. VanRaay, T. J., et al., frizzled 9 is expressed in neural precursor cells in the developing neural tube. Dev. Genes Evol., 2001. 211: p. 453-457.
43. Marti, E. and P. Bovolenta, Sonic Hedgehog in CNS Development: One Signal, Multiple Outputs. Trends Neurosci., 2002. 25: p. 89-96.
44. Fedtsova, N., R. Perris, and E. E. Turner, Sonic Hedgehog Regulates the Position of the Trigeminal Ganglia. Dev. Biol., 2003. 261: p. 456-469.
45. Zhang, J.-M., R. Hoffmann, and M. Sieber-Blum, Mitogenic and Anti-Proliferative Signals for Neural Crest Cells and the Neurogenic Action of TGF-β1. Dev. Dyn., 1997. 208: p. 375-386.
46. Ota, M. and K. Ito, Induction of Neurogenin-1 Expression by Sonic Hedgehog: Its role in Development of Trigeminal Sensory Neurons. Dev. Dyn., 2003. 227: p. 554-551.
47. Perez, S. E., S. Rebelo, and D. J. Anderson, Early Specification of Sensory Neuron Fate Revealed by Expression and Function of Neurogenins in the Chick Embryo. Dev., 1999. 126: p. 1715-1728.
48. Ring, C., J. Hassell, and W. Halfter, Expression Pattern of Collagen IX and Potential Role in the Segmentation of the Peripheral Nervous System. Dev. Biol., 1996. 180: p. 41-53.
49. Perissinotto, D., et al., Avian Neural Crest Cell Migration is Diversely Regulated by the Two Major Hyaluronan-binding Proteoglycans PG-M/Versican and Aggrecan. 137, 2000: p. 2823-2842.
50. Perris, R. and D. Perissinotto, Role of the Extracellular Matrix During Neural Crest Cell Migration. Mech. Dev., 2000. 95: p. 3-21.
51. Gritli-Linde, A., et al., The Whereabouts of a Morphogen: Direct Evidence for Short- and Graded Longe-Range Activity of Hedgehog Signaling Peptides. Dev. Biol., 2001. 236: p. 364-386.
52. Gritli-Linde, A., et al., Shh Signaling within the Dental Epithelium is Necessary for Cell Proliferation, Growth and Polarization. Development, 2002. 129: p. 5323-5337.

1. A method of proliferating a precursor cell comprising modulating the expression of FRS3 in the precursor cell.

2. The method of claim 1 further comprising inducing the precursor cell to differentiate.

3. A method of treating a disorder in a patient characterized by the premature death or malfunction of a specific cell type, the method comprising one of:
(a)proliferating a precursor cell that is the precursor cell for the specific cell type, wherein said proliferating comprises modulating the expression of FRS3 in the precursor cell; and thereafter administering the precursor cell to the patient or
(b) administering to the patient a precursor cell that is the precursor cell for the specific cell type wherein the expression of FRS3 has been modulated in the precursor cell and the precursor cell has increased levels of FRS3 expression relative to the levels of FRS3 expression prior to modulation.

4. (canceled)

5. The method of claim 3 further comprising, in addition to step (a) or step (b), inducing the precursor cell to differentiate prior to administering.

6. The method of claim 1, wherein the precursor cell is chemically or genetically modified to express FRS3 at a greater level than an unmodified precursor cell.

7. The method of claim 3 wherein the disorder is cancer, leukemia, Parkinson's disease, Alzheimer's disease, ALS, CNS damage, spinal cord injury, Multiple Sclerosis, cardiac damage, liver damage, kidney damage, pancreatic damage, retinal damage, intestinal damage, skeletal muscle damage, Muscular dystrophy, lung damage or diabetes.

8. The method of claim 3 wherein the administering comprises surgical implantation or injection.

9. The method of claim 3 wherein the precursor cell has increased levels of endogenous FRS3 as a result of modulation of FRS3.

10. The method of claim 3 wherein the precursor cell has increased levels of exogenous FRS3 as a result of modulation of FRS3.

11. The method of claim 10 wherein the precursor cell is transfected with a vector comprising the exogenous nucleic acid molecule encoding FRS3.

12. The method of claim 11 wherein the vector is a retroviral vector, a lentiviral vector or an Adenoviral vector.

13. The method of claim 1 wherein the precursor cell further comprises a nucleic acid encoding a therapeutic transgene or a therapeutic peptide.

14. The method of claim 1 further comprising treating the precursor cell with an additional growth factor.

15. The method of claim 14 wherein the additional growth factor is fibroblast growth factor or a neurotrophin.

16. The method of claim 1 wherein the FRS3 is human FRS3 or mouse FRS3.

17. The method of claim 16 wherein the FRS3 is (i) a protein comprising the amino acid sequence of SEQ ID NO.:1 or SEQ ID NO.:2; (ii) a fragment of SEQ ID NO.:1 or SEQ ID NO.:2, the fragment possessing substantially the ability to induce or enhance proliferation of the precursor cell or to cause the precursor cell to respond to growth factors; or (iii) an amino acid sequence possessing 90% identity to SEQ ID NO.:1 or SEQ ID NO.:2 and possessing substantially the ability to induce or enhance proliferation of the precursor cell or to cause the precursor cell to respond to growth factors.

18. The method of claim 1 wherein the precursor cell is derived from neuronal tissue, peripheral blood, bone marrow, cardiac muscle, liver, retina, skeletal muscle, kidney, pancreatic, spleen, intestinal, lung, skin, umbilical cord cells including umbilical vein endothelial cells, or embryonic cells including embryonic stem cells.

19. A precursor cell expressing FRS3 wherein the expression of FRS3 has been modulated in the precursor cell and the precursor cell has increased levels of FRS3 expression relative to the levels of FRS3 expression prior to modulation.

20. The precursor cell of claim 19 further comprising a nucleic acid encoding a therapeutic transgene or a therapeutic peptide.

21. The precursor cell of claim 19 wherein the FRS3 is human FRS3 or mouse FRS3.

22. The precursor cell of claim 21 wherein the FRS3 is (i) a protein comprising the amino acid sequence of SEQ ID NO.:1 or SEQ ID NO.:2; (ii) a fragment of SEQ ID NO.:1 or SEQ ID NO.:2, the fragment possessing substantially the ability to induce or enhance proliferation of the precursor cell or to cause the precursor cell to respond to growth factors; or (iii) an amino acid sequence possessing 90% identity to SEQ ID NO.:1 or SEQ ID NO.:2 and possessing substantially the ability to induce or enhance proliferation of the precursor cell or to cause the precursor cell to respond to growth factors.

23. The precursor cell of claim 19 wherein the precursor cell is derived from neuronal tissue, peripheral blood, bone marrow, cardiac muscle, liver, retina, skeletal muscle, kidney, pancreatic, spleen, intestinal, lung, skin, umbilical cord cells including umbilical vein endothelial cells, or embryonic cells including embryonic stem cells.

24. A progeny cell of the precursor cell of claim 9.

25. A pharmaceutical composition comprising the precursor cell of claim 19 and a pharmaceutically acceptable carrier.

26. The method of claim 1 wherein modulating the expression of FRS3 comprises increasing expression levels of endogenous FRS3 in the precursor cell.

27. The method of claim 1 wherein modulating the expression of FRS3 comprises expressing in the precursor cell an exogenous nucleic acid molecule encoding FRS3.

28. The method of claim 27 wherein modulating the expression of FRS3 comprises transfecting the precursor cell with a vector comprising the exogenous nucleic acid molecule encoding FRS3.

29. The method of claim 28 wherein the vector is a retroviral vector, a lentiviral vector or an Adenoviral vector.

30. The method of claim 3, wherein the precursor cell is chemically or genetically modified to express FRS3 at a greater level than an unmodified precursor cell.

31. The method of claim 3 wherein the precursor cell further comprises a nucleic acid encoding a therapeutic transgene or a therapeutic peptide.

32. The method of claim 3 further comprising treating the precursor cell with an additional growth factor.

33. The method of claim 32 wherein the additional growth factor is fibroblast growth factor or a neurotrophin.

34. The method of claim 3 wherein the FRS3 is human FRS3 or mouse FRS3.

35. The method of claim 34 wherein the FRS3 is (i) a protein comprising the amino acid sequence of SEQ ID NO.:1 or SEQ ID NO.:2; (ii) a fragment of SEQ ID NO.:1 or SEQ ID NO.:2, the fragment possessing substantially the ability to induce or enhance proliferation of the precursor cell or to cause the precursor cell to respond to growth factors; or (iii) an amino acid sequence possessing 90% identity to SEQ ID NO.:1 or SEQ ID NO.:2 and possessing substantially the ability to induce or enhance proliferation of the precursor cell or to cause the precursor cell to respond to growth factors.

36. The method of claim 3 wherein the precursor cell is derived from neuronal tissue, peripheral blood, bone marrow, cardiac muscle, liver, retina, skeletal muscle, kidney, pancreatic, spleen, intestinal, lung, skin, umbilical cord cells including umbilical vein endothelial cells, or embryonic cells including embryonic stem cells.

37. A pharmaceutical composition comprising the progeny cell of claim 24 and a pharmaceutically acceptable carrier.
